(19) United States
(12) Patent Application Publication
(10) Pub. No.: US 2024/0424150 A1
(43) Pub. Date: Dec. 26, 2024

(54) LEAD (II)-CONTAINING NANOPARTICLES AS X-RAY CONTRAST AGENTS DISPERSED IN ALGINATE GELS

(71) Applicants: Saint Louis University, St. Louis, MO (US); The Ohio State University, Columbus, OH (US)

(72) Inventors: Paul Andrew Jelliss, St. Louis, MO (US); Steven Wayne Buckner, St. Louis, MO (US); Mohammad Salauddin Kader, St. Louis, MO (US); Sarah Howe McBride-Gagyi, Westerville, OH (US); Kyan Dang Ho, Eureka, MO (US); Madhushika E. Gamage, St. Louis, MO (US)

(21) Appl. No.: 18/750,393

(22) Filed: Jun. 21, 2024

Related U.S. Application Data
(60) Provisional application No. 63/509,591, filed on Jun. 22, 2023.

(51) Int. Cl. A61K 49/04; A61K 47/10; A61K 47/22; A61K 47/36

(52) U.S. Cl. CPC A61K 49/0423; A61K 47/10; A61K 47/22; A61K 47/36

(57) ABSTRACT

Disclosed are capped abellaite (NaPb₂(CO₃)₂OH), hydrocerussite (2PbCO₃—Pb(OH)₂), lead (II) carbonate (PbCO₃), lead (II) tungstate (PbWO₄), bismuth oxide (Bi₂O₃), and combinations thereof nanoparticles that are dispersed within an aqueous gelling solution to produce stable gels and function as an injectable contrast agent for vascular imaging. The contrast agent has good radioopacity, is inexpensive to produce, and is safe to handle. This provides a new method to image the fine vasculature of biological systems.

### CROSS-REFERENCE TO RELATED APPLICATIONS

This application claims the benefit of priority to U.S. Application Ser. No. 63/509,591 filed on Jun. 22, 2023, the contents of which are herein incorporated by reference in their entirety.

### BACKGROUND OF THE DISCLOSURE

The present disclosure relates generally vascular imaging. In particular, the present disclosure is directed to nanoparticle-containing composite gels for improved imaging the vasculature of biological systems using X-ray techniques and synthesis of abellaite (NaPb₂(CO₃)₂OH), hydrocerussite (2PbCO₃—Pb(OH)₂), bismuth oxide (Bi₂O₃), lead (II) carbonate, and lead (II) tungstate nanoparticles as X-ray contrast agents dispersed in alginate gels.

X-ray imaging is a very important technique to distinguish the vascular network from tissues with similar or low X-ray attenuation. The choice of contrast agent is critical for this technique. An unstable contrast agent can easily dissociate or leach from the host matrices, which can lead to fuzzy imaging and misdiagnosis. Also, the lack of inherent radiopaque agents has severely hindered the imaging technique.

Barium sulfate particles are a commonly used contrast agent clinically for gastrointestinal imaging, and are more radiodense than bone. However, commercially available barium sulfate particles are already approaching or larger than the size of capillaries and tend to bind in solution to make even larger objects. Thus, the particles clog at the capillary level, never enter the venous system, and the pressure ultimately ruptures the aorta.

Bismuth-containing nanoparticles have many applications including biomedical applications. Bismuth is useful as the largest atomic mass nucleus that is “effectively” stable (lifetime>>estimated age of the universe). Bismuth and its compounds are generally non-toxic (with exceptions), which is unusual for the heavy metals in this portion of the periodic table. This high mass makes it a very useful element for X-ray imaging methods. Thus, the particles clog at the capillary level, never enter the venous system, and the pressure ultimately ruptures the aorta.

Accordingly, there exists a need for new and alternative contrast agents for imaging vasculature, particularly vasculature within and in proximity to bone.

### BRIEF DESCRIPTION OF THE DISCLOSURE

In one aspect, the present disclosure is directed to an injectable contrast agent comprising: a nanoparticle, the nanoparticle comprising a core comprising at least one of an abellaite (NaPb₂(CO₃)₂OH) core, a hydrocerussite (2PbCO₃—Pb(OH)₂) core, a lead (II) carbonate (PbCO₃) core, a lead (II) tungstate (PbWO₄) core, a bismuth oxide (Bi₂O₃) core, and combinations thereof; and a capping agent comprising at least one of an oligomer and a polymer; and a gel precursor solution.

In one aspect, the present disclosure is directed to a method for post mortem imaging vasculature of a subject, the method comprising: introducing into the subject's vasculature an injectable contrast agent, the injectable contrast agent comprising a nanoparticle, the nanoparticle comprising a core comprising at least one of an abellaite (NaPb₂(CO₃)₂OH) core, a hydrocerussite (2PbCO₃—Pb(OH)₂) core, a lead (II) carbonate (PbCO₃) core, a lead (II) tungstate (PbWO₄) core, a bismuth oxide (Bi₂O₃) core, and combinations thereof; and a capping agent comprising at least one of an oligomer and a polymer; and a gel precursor solution, wherein the nanoparticle is dispersed in the gel precursor solution; and imaging the subject using an X-ray technique.

In one aspect, the present disclosure is directed to an abellaite (NaPb₂(CO₃)₂OH) nanoparticle comprising an abellaite (NaPb₂(CO₃)₂OH) core wherein the core ranges in diameter from about 1 nm to about 100 nm.

In one aspect, the present disclosure is directed to a hydrocerussite (2PbCO₃—Pb(OH)₂) nanoparticle comprising an hydrocerussite (2PbCO₃—Pb(OH)₂) core wherein the core ranges in diameter from about 1 nm to about 100 nm.

In one aspect, the present disclosure is directed to a bismuth oxide (Bi₂O₃) nanoparticle comprising a bismuth oxide (Bi₂O₃) core ranging in diameter from about 0.5 nm to about 5 nm.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 depicts a powder x-ray diffraction (PXRD) pattern of abellaite (NaPb₂(CO₃)₂OH) (ICDD #01-074-3175). The Miller indices of the five most prominent peaks for each pattern are labeled. Average Scherrer width particle sizes (25±5 nm).

FIG. 2(a) depicts TEM of abellaite (NaPb₂(CO₃)₂OH) NPs capped with TEG. Scale bar 100 nm.

FIG. 2(b) depicts a size distribution histogram of abellaite NPs (40±5 nm).

FIG. 3(a) depicts a suspension of homogenous abellaite NPs in pure liquid TEG.

FIG. 3(b) depicts a dynamic light scattering (DLS) particle size plot of abellaite NPs in liquid TEG (40±5 nm).

FIG. 4 depicts a PXRD pattern of PbWO₄(ICDD #01-086-0843). The Miller indices of the five most prominent peaks for each pattern are labeled. Average Scherrer width particle sizes (50±5 nm).

FIG. 5(a) depicts TEM of PbWO₄ NPs capped with TEG. Scale bar 100 nm.

FIG. 5(b) depicts a size distribution histogram of PbWO₄ NPs (65±5 nm).

FIG. 6(a) depicts a homogenous suspension of PbWO₄ NPs in pure liquid TEG.

FIG. 6(b) depicts a DLS particle size plot of PbWO₄ NPs in liquid TEG (60±5 nm).

FIG. 7 depicts an abellaite NPs-GDL-alginate nanocomposite hydrogel cylinder (50 mL).

FIG. 8 depicts stress, kPa vs strain for abellaite NPs-GDL-alginate nanocomposite gel (abellaite NP, solid line) compared with BaSO₄ BNPs-CaCO₃ CNPs-GDL-alginate nanocomposite gel (BNP, dashed line),

FIG. 9(a) depicts a MicroCT scout view of the abellaite NPs-GDL-alginate nanocomposite gels.

FIG. 9(b) depicts a MicroCT scanning of the abellaite NPs-GDL-alginate nanocomposite gels at different concentrations (0.10-0.40 g/mL) of BNPs.

FIG. 10 depicts a calibration curve (MicroCT radiodensity measured in Houndsfields units as a function of nanoparticle concentration) comparison of abellaite NPs-GDL-alginate nanocomposite gel (LNP, solid line) with BaSO₄ BNPs-CaCO₃ CNPs-GDL-alginate nanocomposite gel (BNP, dashed line).

FIGS. 11(a)-11(e) depict PXRD patterns for abellaite NPs (ICDD #01-074-3175) synthesized from Na₂CO₃ (FIG. 11(a)), hydrocerussite NPs (ICDD #01-073-4362, hydrocerussite, syn) synthesized from K₂CO₃ (FIG. 11(b)), cerussite NPs, (ICDD #01-083-3087 (lead (II) carbonate) synthesized from (NH₄)₂CO₃ (FIG. 11(c), NaHCO₃ (FIG. 11(d)), and NH₄HCO₃ FIG. 11(e).

FIGS. 12(a)-12(d) depict a TEM micrograph of abellaite NPs capped with TEG (FIG. 12(a)) and size distribution histograms of abellaite NPs (FIG. 12(b)), hydrocerussite NPs (FIG. 12(c)), and cerussite NPs (FIG. 12(d)).

FIG. 13 is a graph depicting averaged DLS size plot of abellaite NPs dispersed in liquid TEG across three trials. The plot shows a peak at 267.2 nm with a standard deviation of 71.9 nm.

FIG. 14 is a graph depicting the ATR-FTIR spectra for abellaite, hydrocerussite, and cerussite nanoparticles.

FIG. 15 is a graph depicting DSC (blue) and TGA(green) curves for abellaite NPs from 50 to 1000° C. with a heating rate of 10° C./min.

FIG. 16 depicts transformation of d-(+)-gluconic acid δ-lactone (GDL) to gluconic acid in the presence of water.

FIG. 17(a) depicts the Egg-box model of metal dication-alginate binding.

FIG. 17(b) is a photograph of an alginate hydrogel disc comprising the abellaite NP nanocomposite.

FIG. 18 is a graph depicting storage modulus vs time for abellaite NPs-GDL-alginate composite and BNPs-CNPs-GDL-alginate composite gels.

FIG. 19 is a graph depicting stress-strain curve for abellaite NPs-GDL-alginate composite and BNPs-CNPs-GDL-alginate composite gels.

FIG. 20 depicts micro-CT scanning at different X-ray intensity thresholds (300-700 permilles).

### DETAILED DESCRIPTION

Unless defined otherwise, all technical and scientific terms used herein have the same meaning as commonly understood by one of ordinary skill in the art to which the disclosure belongs.

In one aspect, the present disclosure is directed to an injectable contrast agent comprising: a nanoparticle, the nanoparticle comprising a core comprising at least one of an abellaite (NaPb₂(CO₃)₂OH) core, a hydrocerussite (2PbCO₃—Pb(OH)₂) core, a lead (II) carbonate (PbCO₃) core, a lead (II) tungstate (PbWO₄) core, a bismuth oxide (Bi₂O₃) core, and combinations thereof; and a capping agent comprising at least one of an oligomer and a polymer; and a gel precursor solution.

Suitable nanoparticle core materials include abellaite (NaPb₂(CO₃)₂OH)) cores, hydrocerussite (2PbCO₃—Pb(OH)₂) cores, lead (II) carbonate (PbCO₃) cores, lead (II) tungstate (PbWO₄) cores, bismuth oxide (Bi₂O₃) cores, and combinations thereof. Particularly suitable core materials include abellaite cores, hydrocerussite cores, bismuth oxide cores, and combinations thereof.

The nanoparticle core is capped with a capping agent including oligomers, polymers, and combinations thereof as described herein. As used herein, “capping” and “capped” by the oligomer and polymer capping agents provides a homogenous but semi-permeable layer, tightly bound around the ionic nanoparticle core. The underlying ionic structure is thermodynamically stable, but capping imparts favorable characteristics to the nanoparticle core such as permitting dispersion in aqueous media. Without being bound by theory, the capping oligomer and polymer binds to the nanoparticle core surface via predominantly ion-dipole interactions. Suitable capping agents for capping the core material include oligomers, polymers, and combinations thereof. Tetraethylene glycol (TEG) is a particularly suitable capping agent. Other suitable polymers include polyethylene glycol (PEG) including PEG-600 to PEG-500,000, polyvinyl alcohol (PVA), polyacrylic acid (PAA), polyacrylamide (PAM), poly(sodium 4-styrenesulfonate) (PSS), for example. Chain-end modified PEG can also be used such as, for example, those with amino NH₂ groups and hydroxyl OH groups. Low molecular weight oligoethers (oligomers related to PEG) include triethethylene glycol and tetraethylene glycol (TEG) are also suitable capping agents.

The gel precursor solution includes a material that forms a gel (a “gel forming material”) upon crosslinking. So that the contrast agent is injectable, the gel precursor solution provides a matrix that carries the oligomer- and polymer-capped core nanoparticles to a local tissue site in the subject. Upon gelation, the gel precursor solution forms a gel and retains the nanoparticles in the local tissue site. Suitable gel forming materials include alginate. Other suitable gelling agents include those that do not require heating above 50° C., for example, chitosan, silicon-based sol-gels, and gellan gum. Suitably, the amount of alginate in the final alginate composition of the resulting contrast agent ranges from about 0.05% (wt/wt) to about 5% (wt/wt). Preferably, the amount of alginate ranges from about 0.1% (wt/wt) to about 2% (wt/wt).

In addition to contrast agents where all of the core particles are prepared using the same core material and have the same capping agent, different combinations of materials are also contemplated herein. Different combinations of materials include combinations of different core materials (e.g., an abellaite (NaPb₂(CO₃)₂OH) core, a hydrocerussite (2PbCO₃—Pb(OH)₂) core, a lead (II) carbonate (PbCO₃) core, a lead (II) tungstate (PbWO₄) core, and a bismuth oxide (Bi₂O₃) core) with the same or different capping oligomers and capping polymers (e.g., PEG, TEG). For example abellaite (NaPb₂(CO₃)₂OH) cores, hydrocerussite (2PbCO₃—Pb(OH)₂) cores, lead (II) carbonate (PbCO₃) cores, lead (II) tungstate (PbWO₄) cores, and/or bismuth oxide (Bi₂O₃) cores capped with PEG can be combined with abellaite (NaPb₂(CO₃)₂OH)) cores, hydrocerussite (2PbCO₃—Pb(OH)₂) cores, lead (II) carbonate (PbCO₃) cores, lead (II) tungstate (PbWO₄) cores, and/or bismuth oxide (Bi₂O₃) cores capped with TEG, which are then combined with the gel precursor solution to prepare the injectable contrast agent/gel matrix. Similarly, combinations of cores capped with PEG can be combined with cores capped with TEG, which are then combined with the gel precursor solution to prepare the injectable contrast agent/gel matrix. Similarly, one type of core material capped with PEG can be combined with other core materials capped with PEG, which are then combined with the gel precursor solution to prepare the injectable contrast agent/gel matrix. The final radiopacity of the injectable contrast agent gel can be controlled by adjusting the concentration of capped nanoparticles in the gel precursor solution. The concentration of capped nanoparticles in the final injectable contrast agent gel can range from about 0.05 g/mL to about 1 g/mL for most practical imaging purposes.

Suitably, the amount of nanoparticle core in the contrast agent can range from about 0.05 g/mL to about 1 g/mL. Suitably, the diameter of the core particles ranges from about 1 nm to about 500 nm, including from about 1 nm to about 10 nm. A particularly suitable particle size range for Bi₂O₃ nanoparticle core ranges from about 1 nm to about 5 nm. Particle size can be determined by methods known to one skilled in the art such as by dynamic light scattering (DLS), powder X-ray diffraction (PXRD), transmission electron microscopy (TEM), and combinations thereof, for example.

The nanoparticle cores are capped with a capping agent resulting in the formation of the core-shell nanoparticles. As discussed herein, capping of the core particles with a capping agent stabilizes the core and also confers water dispersibility to the system. The amount of capping agent used in the preparative solution for capping ranges from about 0.05% (v/v) to about 80% (v/v). An aqueous capping agent preparative solution is used to synthesize the nanoparticle composites. The amount of capping agent for the capped cores can be determined by elemental analysis.

The gel precursor solution of the injectable contrast agent includes a material that forms a gel (a ‘gel-forming material’) upon cross-linking. A particularly suitable gel-forming material is alginate. The amount of alginate in the final gel composition of the resulting contrast agent can range from about 0.01% (w/w) to about 2% (w/w). Preferably, the amount of alginate is about 1% (w/w). Other suitable gelling agents include those that do not require heating above 50° C., for example, chitosan, silicon-based sol-gels, and gellan gum. The gel precursor solution of the injectable contrast agent can further include an initiator to adjust the pH of the solution and control gel formation rate. Suitable initiators to control gelation include glucono-δ-lactone (GDL) in combination with lead (II) carbonate-containing and/or other metal (II) carbonates (MCO₃ where M²⁺=Cu²⁺, Zn²⁺, or Ca²⁺ ions), and soluble calcium salts such as CaCl₂.

The injectable contrast agent can further include at least a second polymer to improve gel stiffness. Suitable second polymers include polyacrylic acid (PAA), polyvinyl alcohol, chitosan, and combinations thereof.

The injectable contrast agent can further include a chelating gelation control reagent for controlling gel formation of the contrast agent. Suitable chelating gelation control reagents include ethylenediaminetetraacetic acid (EDTA) and/or glucono-δ-lactone (GDL). Other suitable chelating gelation control reagents include: nitrilotriacetic acid (NTA), trans-1,2-diaminocylcohexanetetraacetic acid (DCTA), diethlyenetriaminepentaacetic acid (DTPA), and bis(aminoethyl)glycolether-N,N,N′,N′-tetraacetic acid (EGTA).

The polymer- or oligomer-capped core nanoparticles are effectively trapped within a rigid gel that forms from an aqueous solution by crosslinking Pb²⁺ ions with gelling polymers such as alginate.

In one aspect, the present disclosure is directed to a method for imaging vasculature of a subject. The method includes: introducing into the subject's vasculature an injectable contrast agent, the injectable contrast agent comprising a nanoparticle, the nanoparticle comprising a core comprising at least one of an abellaite (NaPb₂(CO₃)₂OH)) core, a hydrocerussite (2PbCO₃—Pb(OH)₂) core, a lead (II) carbonate (PbCO₃) core, a lead (II) tungstate (PbWO₄) core, a bismuth oxide (Bi₂O₃) core, and combinations thereof; and a capping agent comprising at least one of an oligomer and a polymer; and a gel precursor solution, wherein the nanoparticle is dispersed in the gel precursor solution; and imaging the subject using an X-ray technique.

In one aspect, the present disclosure is directed to a method for post mortem imaging vasculature of a subject. The method includes: introducing into the subject's vasculature an injectable contrast agent, the injectable contrast agent including a nanoparticle, the nanoparticle including a core comprising at least one of an abellaite (NaPb₂(CO₃)₂OH) core, a hydrocerussite (2PbCO₃—Pb(OH)₂) core, a lead (II) carbonate (PbCO₃) core, a lead (II) tungstate (PbWO₄) core, a bismuth oxide (Bi₂O₃) core, and combinations thereof; and a capping agent including at least one of an oligomer and a polymer; and a gel precursor solution, wherein the nanoparticle is dispersed in the gel precursor solution; and imaging the subject using an X-ray technique.

The injectable contrast agent has a gelation time ranging from about 5 minutes to about 48 hours.

Suitable materials for the nanoparticle core are described herein. In particular, abellaite (NaPb₂(CO₃)₂OH) cores, hydrocerussite (2PbCO₃—Pb(OH)₂) cores, lead (II) carbonate (PbCO₃) cores, lead (II) tungstate (PbWO₄) cores, bismuth oxide (Bi₂O₃) cores, and combinations thereof.

Suitable capping agents for capping the core material include oligomers, polymers, and combinations thereof. Tetraethylene glycol (TEG) is a particularly suitable capping agent. Other suitable polymers include polyethylene glycol (PEG) including PEG-600 to PEG-500,000, polyvinyl alcohol (PVA), polyacrylic acid (PAA), polyacrylamide (PAM), poly(sodium 4-styrenesulfonate) (PSS), for example. Chain-end modified PEG can also be used such as, for example, those with amino NH₂ groups and hydroxyl OH groups. Low molecular weight oligoethers (oligomers related to PEG) include triethethylene glycol and tetraethylene glycol (TEG) are also suitable capping agents.

Suitable gel forming materials are described herein.

Suitable subjects include animals. Particularly suitable subjects are humans (cadavers), primates, mice, rats, rabbits, birds, and other animals, particularly laboratory research animals. Suitable subjects can be animals suffering from a disease or disorder that would require the use of the contrast agent to image a tissue site. Introducing the contrast agent into the subject's vasculature is through perfusion, particularly postmortem perfusion. It is particularly suitable to use the contrast agent of the present disclosure in human autopsies to determine if a vascular event (e.g., blocked arteries; embolism; etc.) resulted in cause of death.

The gelation rate of the injectable contrast agent can be controlled. Gelation rate is controlled to allow sufficient time for the contrast agent to be introduced into the vasculature of the subject before crosslinking into a gel. Thus, the method can further include adding a chelating gelation control reagent for controlling gelation time to one of the crosslinker and the gel precursor solution. Gelation rates can range from about 10 seconds to about 48 hours at a temperature ranging from about 5° C. to about 40° C. and within a pH ranging from about 5.0 to about 7, including from about 5.5 to about 6.5. The ratio of Pb²⁺ ions from the abellaite (NaPb₂(CO₃)₂OH) cores, hydrocerussite (2PbCO₃—Pb(OH)₂) cores, lead (II) carbonate (PbCO₃) cores, and/or lead (II) tungstate (PbWO₄) cores to GDL can be used to control gelation time. Gelation time can be controlled by adding a 0.1 M solution of EDTA to the gel precursor solution, for example. Suitable alternatives to EDTA include PbCO₃ or CaCO₃ in combination with glucono-δ-lactone. The ratio of Pb²⁺ or Ca²⁺ ions to GDL can be used to control gelation time. In an exemplary gel composition, the amounts include 0.5% w/w nanoparticle and 1.5% w/w GDL (the mass % for the whole gel including nanoparticles, GDL, Na-alginate, and also water). A suitable range is from about 0.1% w/w to about 1.0% w/w nanoparticles and also from about 0.3% w/w to about 3.0% w/w GDL. The gelation rate can range from about 5 minutes to about 48 hours.

Other suitable chelating gelation control reagents include: nitrilotriacetic acid (NTA), trans-1,2-diaminocylcohexanetetraacetic acid (DCTA), diethlyenetriaminepentaacetic acid (DTPA), and bis(aminoethyl)glycolether-N,N,N′,N′-tetraacetic acid (EGTA).

The radioopacity of the injectable contrast agent can be controlled by adjusting the concentration of capped nanoparticles in the gel precursor solution. Suitably, the concentration of capped nanoparticles ranges from about 0.05 g/mL to about 1 g/mL.

Suitably, the amount of nanoparticle core in the contrast agent can range from about 0.05 g/mL to about 1 g/mL. Suitably, the diameter of the core particles ranges from about 1 nm to about 500 nm, including from about 1 nm to about 10 nm, and including from about 1 nm to about 5 nm. A particularly suitable particle size range for Bi₂O₃ ranges from about 1 nm to about 5 nm. Particle size can be determined by methods known to one skilled in the art such as by dynamic light scattering (DLS), powder X-ray diffraction (PXRD), transmission electron microscopy (TEM), and combinations thereof, for example.

In one aspect, the present disclosure is directed to an abellaite (NaPb₂(CO₃)₂OH) nanoparticle comprising an abellaite (NaPb₂(CO₃)₂OH) core wherein the core ranges in diameter from about 1 nm to about 100 nm. In another embodiment, the abellaite nanoparticle further includes a capping agent.

Suitable capping agents for capping the core material include oligomers, polymers, and combinations thereof. Tetraethylene glycol (TEG) is a particularly suitable capping agent. Other suitable polymers include polyethylene glycol (PEG) including PEG-600 to PEG-500,000, polyvinyl alcohol (PVA), polyacrylic acid (PAA), polyacrylamide (PAM), poly(sodium 4-styrenesulfonate) (PSS), for example. Chain-end modified PEG can also be used such as, for example, those with amino NH₂ groups and hydroxyl OH groups. Low molecular weight oligoethers (oligomers related to PEG) include triethethylene glycol and tetraethylene glycol (TEG) are also suitable capping agents.

In one aspect, the present disclosure is directed to a hydrocerussite (2PbCO₃—Pb(OH)₂) nanoparticle comprising an hydrocerussite (2PbCO₃—Pb(OH)₂) core wherein the core ranges in diameter from about 1 nm to about 100 nm. In another embodiment, the hydrocerussite nanoparticle further includes a capping agent.

Suitable capping agents for capping the core material include oligomers, polymers, and combinations thereof. Tetraethylene glycol (TEG) is a particularly suitable capping agent. Other suitable polymers include polyethylene glycol (PEG) including PEG-600 to PEG-500,000, polyvinyl alcohol (PVA), polyacrylic acid (PAA), polyacrylamide (PAM), poly(sodium 4-styrenesulfonate) (PSS), for example. Chain-end modified PEG can also be used such as, for example, those with amino NH₂ groups and hydroxyl OH groups. Low molecular weight oligoethers (oligomers related to PEG) include triethethylene glycol and tetraethylene glycol (TEG) are also suitable capping agents.

In one aspect, the present disclosure is directed to a bismuth oxide (Bi₂O₃) nanoparticle comprising a bismuth oxide (Bi₂O₃) core ranging in diameter from about 0.5 nm to about 5 nm. In another embodiment, the bismuth oxide (Bi₂O₃) nanoparticle further includes a capping agent.

Suitable capping agents for capping the core material include oligomers, polymers, and combinations thereof. Tetraethylene glycol (TEG) is a particularly suitable capping agent. Other suitable polymers include polyethylene glycol (PEG) including PEG-600 to PEG-500,000, polyvinyl alcohol (PVA), polyacrylic acid (PAA), polyacrylamide (PAM), poly(sodium 4-styrenesulfonate) (PSS), for example. Chain-end modified PEG can also be used such as, for example, those with amino NH₂ groups and hydroxyl OH groups. Low molecular weight oligoethers (oligomers related to PEG) include triethethylene glycol and tetraethylene glycol (TEG) are also suitable capping agents.

### EXAMPLES

### Example 1

### Abellaite Nanoparticle Synthesis

Nanoparticles were synthesized according to the following protocols and characterized using powder X-ray diffraction (PXRD), transmission electron microscopy (TEM), dynamic light scattering (DLS), and thermogravimetric analysis/differential scanning calorimetry (DSC/TGA).

### Abellaite@TEG LNP Synthesis

### Materials

Sodium carbonate (Na₂CO₃)
Lead (II) nitrate (Pb(NO₃)₂)
Tetraethylene glycol (TEG)

Procedure: PbCO₃ nanoparticle synthesis was carried out by the following arrested precipitation reaction. To prevent bulk PbCO₃ formation, a capping agent (TEG) was added during the precipitation process. The amounts listed are for a typical synthesis and are not limiting.

20 g of Na₂CO₃ was dissolved in a 500 mL volumetric flask using DI water to afford an 0.38 M solution. 20 g of Pb(NO₃)₂ solution was dissolved in another 500 mL volumetric flask using DI water to afford an 0.12 M solution. The 500 mL Na₂CO₃ solution was transferred to a 2000 mL beaker and approximately 100 mL of the TEG was added to the solution with continuous stirring using a glass rod. The previously prepared Pb(NO₃)₂ solution was added dropwise to the TEG/Na₂CO₃ solution with continuous stirring and sonication using a sonic dismembrator at room temperature. The sonic dismembrator was tuned so that it was highly pitched and visibly mixing the solution (this was readjusted throughout the addition). Drops were added closest to the sonic dismembrator probe at a rate of 2 drops per second. After each full dispense of the pipette, the beaker was moved so that the probe reached the edges in a circular twisting motion and was then returned to the middle of the beaker. Once all of the solutions were combined under the sonic dismembrator, a watch glass was placed on top of the beaker holding the suspended nanoparticles. The suspension of abellaite@TEG NPs was centrifuged (15 min, 4000 rpm) and then rinsed three times with water, centrifuging after each wash. The wet solid was dried in the oven at 80-100° C. for about 10-12 hours to obtain 18.50 g white abellaite @TEG nanoparticle powder.

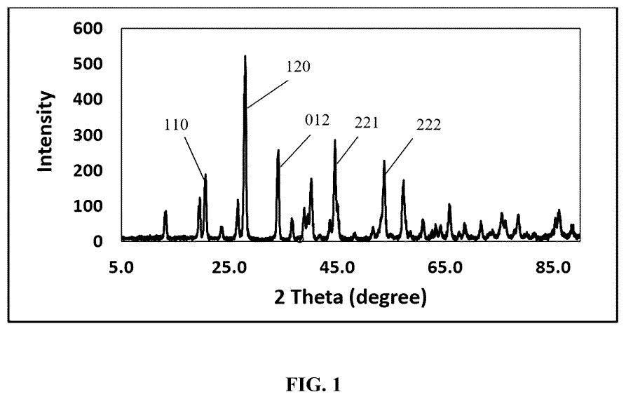

PXRD: Measured pattern in FIG. 1 confirmed as abellaite phase (ICDD #01-074-3175) with average nanoparticle (crystallite core) diameter of 25±5 nm (based on Scherrer peak width calculations from the 5 strongest peaks).

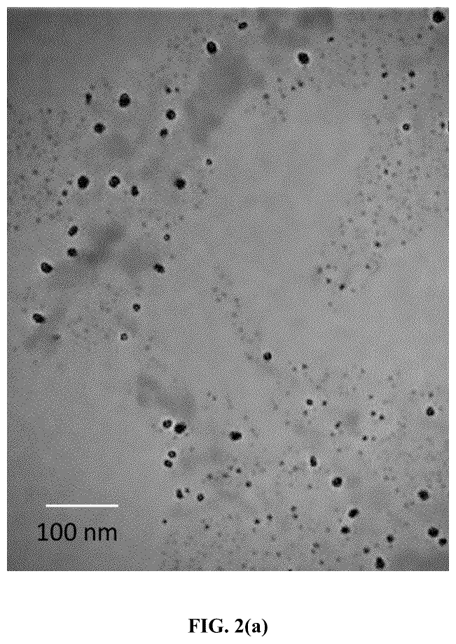

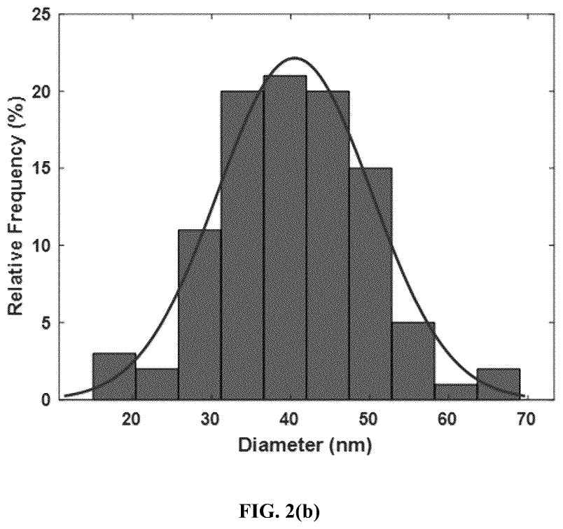

TEM: Micrograph in FIG. 2(a) shows roughly spherical nanoparticles averaging 40±5 nm in diameter. The distribution in sizes for ca. 100 nanoparticles is shown in the histogram of FIG. 2(b).

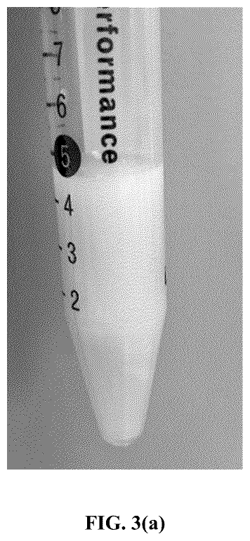

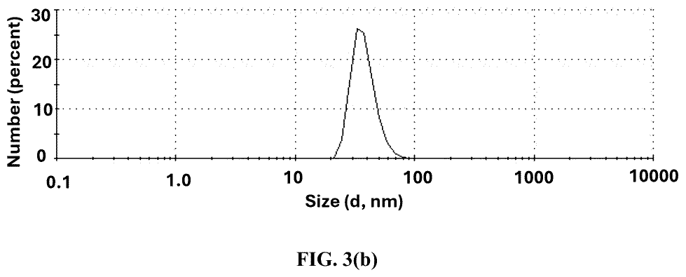

DLS: Nanoparticles dispersed homogenously in TEG as shown in FIG. 3(a) and for diluted suspensions DLS analysis shown in FIG. 3(b) indicated hydrodynamic sizes of 40±5 nm, which closely matches measurements from TEM and indicates little agglomeration of nanoparticles when dispersed in TEG.

DSC/TGA: Thermogram revealed loss of TEG in the range 200-400° C. from ionic nanoparticle cores and permitted estimation of mass composition based on this loss shown in Table 1.

TABLE 1
Abellaite@TEG Nanoparticle Composition

| Material | Mass % (w/w)^{a} | Mass % (w/w) Range^{b} |
|---|---|---|
| Abellaite | 82 | 75-99.95 |
| TEG | 18 | 0.05-25 |

^{a}The above-described synthesis produced abellaite@TEG NPs with this mass % composition (estimated from DSC-TGA of the product).
^{b}Synthetic quantities can be adjusted to produce abellaite@TEG NPs with a composition that falls within each range.

### PbWO₄@TEG LNP Synthesis

### Materials

Sodium tungstate (Na₂WO₄)
Lead (II) nitrate (Pb(NO₃)₂)
Tetraethylene glycol (TEG)

Procedure: PbWO₄ nanoparticle synthesis can be carried out by the following arrested precipitation reaction. To prevent bulk PbWO₄ formation, a capping agent (TEG) was added during the precipitation process. The amounts listed are for a typical synthesis and are not limiting.

10 g of Na₂WO₄ was dissolved in a 100 mL volumetric flask using DI water to afford an 0.34 M solution. The solution may be gently heated on a hot plate to facilitate complete dissolution. 20 g of Pb(NO₃)₂ was dissolved in another 100 mL volumetric flask using DI water to afford an 0.60 M solution. The 100 mL Na₂WO₄ solution was transferred to a 500 mL beaker and approximately 100 mL of the TEG was added to the solution with continuous stirring using a glass rod. The previously prepared Pb(NO₃)₂ solution was added dropwise to the TEG/Na₂WO₄ solution with continuous stirring and sonication using a sonic dismembrator at room temperature. The sonic dismembrator was tuned so that it was highly pitched and visibly mixing the solution (this was readjusted throughout the addition). Drops were added closest to the sonic dismembrator probe at a rate of 2 drops per second. After each full dispense of the pipette, the beaker was moved so that the probe reached the edges in a circular twisting motion and was then returned to the middle of the beaker. Once all of the solutions were combined under the sonic dismembrator, a watch glass was placed on top of the beaker holding the suspended nanoparticles. The suspension of PbWO₄@TEG NPs was centrifuged (15 min, 4000 rpm) and then rinsed three times with water, centrifuging after each wash. The wet solid was dried in the oven at 80-100° C. for about 10-12 hours to obtain 10.50 g white PbWO₄@TEG nanoparticle powder.

### Characterization

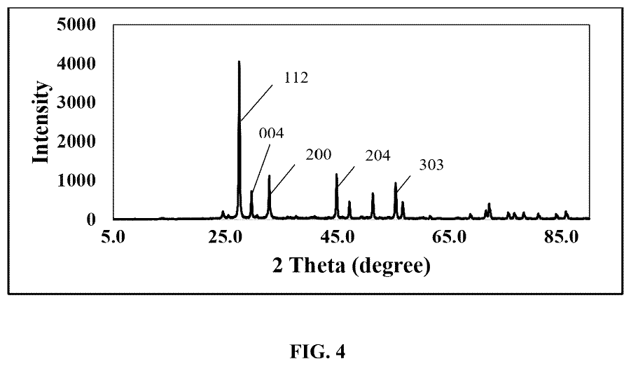

PXRD: Measured pattern in FIG. 4 confirmed as PbWO₄ phase (ICDD #01-086-0843) with average nanoparticle (crystallite core) diameter of 50±5 nm (based on Scherrer peak width calculations from the 5 strongest peaks).

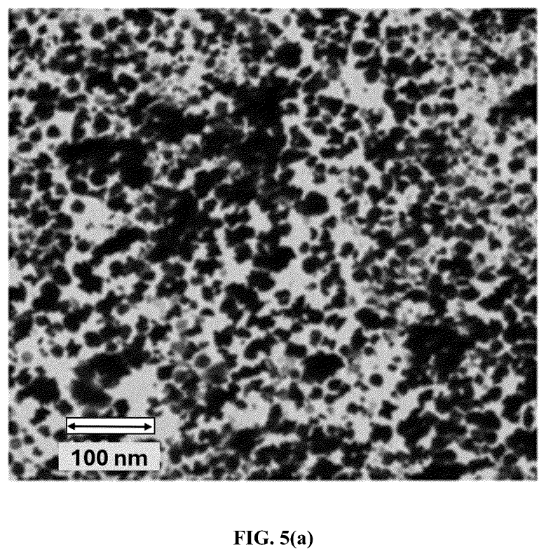

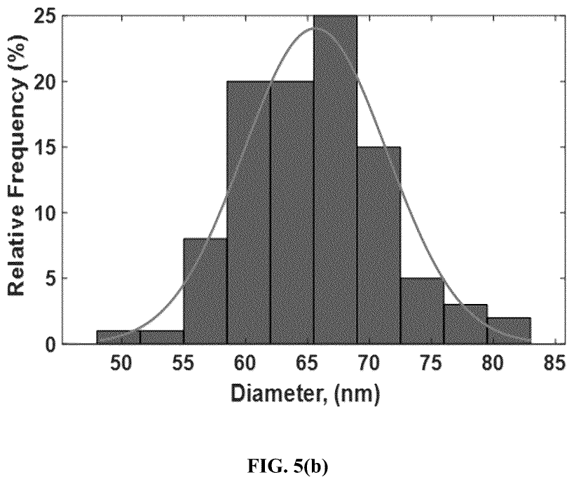

TEM: Micrograph in FIG. 5(a) shows roughly spherical nanoparticles averaging 65±5 nm in diameter. The distribution in sizes for ca. 100 nanoparticles is shown in the histogram of FIG. 5(b).

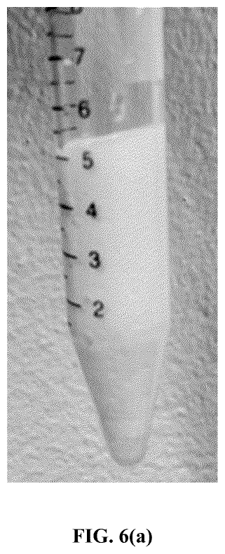

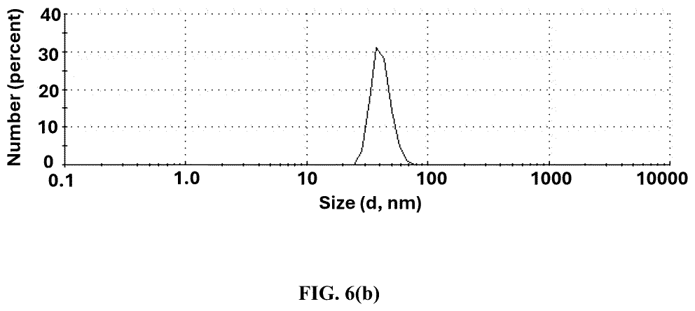

DLS: Nanoparticles dispersed homogenously in TEG as shown in FIG. 6(a) and for diluted suspensions DLS analysis shown in FIG. 6(b) indicated hydrodynamic sizes of 60±5 nm, which closely matches measurements from TEM and indicates little agglomeration of nanoparticles when dispersed in TEG.

DSC/TGA: Thermogram revealed loss of TEG in the range 200-400° C. from ionic nanoparticle cores and permitted estimation of mass composition based on this loss shown in Table 2.

TABLE 2
PbWO₄@TEG Nanoparticle Composition

| Material | Mass % (w/w)^{a} | Mass % (w/w) Range^{b} |
|---|---|---|
| PbWO₄ | 88 | 75-99.95 |
| TEG | 12 | 0.05-25 |

^{a}The above-described synthesis produced PbWO₄@TEG NPs with this mass % composition (estimated from DSC-TGA of the product).
^{b}Synthetic quantities can be adjusted to produce PbWO₄@TEG NPs with a composition that falls within each range.

### Gel Fabrication Procedures

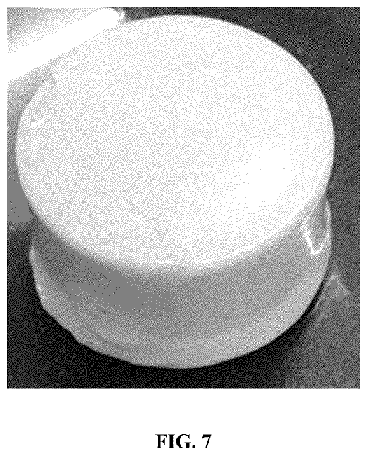

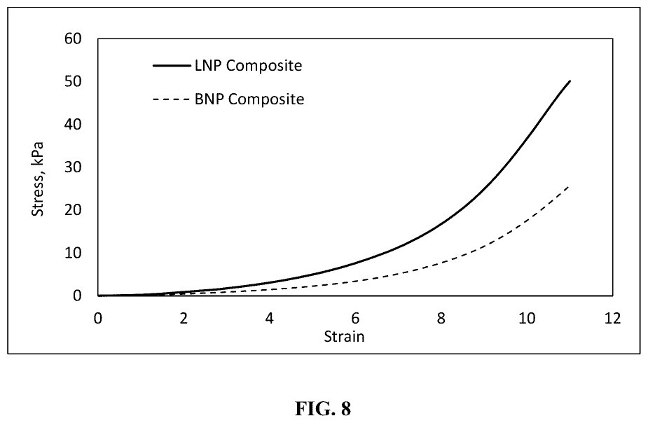

A typical 50 mL cylinder comprised of abellaite LNPs-GDL-alginate nanocomposite gel is shown in FIG. 7. Nanocomposite gel component proportions are shown in Table 3. The superior mechanical strength of the abellaite LNPs-GDL-alginate nanocomposite gel over (recently patented) BaSO₄ BNPs-CaCO₃ CNPs-GDL-alginate nanocomposite gel is demonstrated by rheological analysis in FIG. 8, revealing a peak stress of 50.1 kPa for the abellaite LNPs-GDL-alginate nanocomposite gel versus 18.9 kPa for the BaSO₄ BNPs-CaCO₃ CNPs-GDL-alginate nanocomposite gel.

Synthesis of 50 mL Nanocomposite Gel Cylinder (0.2 g/mL PbCO₃ Only)

### Materials

Sodium alginate (Na-alginate)
abellaite/TEG nanoparticles (abellaite @TEG NPs) powder
Glucono-δ-lactone (GDL)

Procedure: 1.00 g of Na-Alginate was added to 50 mL DI water to prepare a 2% (w/v) Na-alginate solution. Approximately 10 g of the abellaite @TEG NPs powder was dispersed in 20 mL DI water in a 250 mL beaker. The solution was then sonicated for about 5 minutes to uniformly disperse the NPs powder in the solution. 25 mL of the 2% (w/v) Na-alginate solution was added to the abellaite @TEG NPs suspension and mixed thoroughly. 0.50 g of GDL was dissolved in 5 mL DI water. The 5.0 mL GDL solution was added to the 45 mL nanocomposite suspension to initiate crosslinking and mixed thoroughly. Strong acid or strong base may be added dropwise to adjust the pH to within the 5.5-6.5 range to optimize crosslinking for the most effective gelation. Gelling time (40-55 min) was recorded using a stopwatch. GDL, Na-alginate, and abellaite @TEG NPs concentrations may be varied to control the gelling time.

Synthesis of 50 mL Nanocomposite Gel Cylinder (0.2 g/mL Combined PbWO₄/MCO₃ (M=Divalent M₂₊ Cation))

### Materials

Sodium alginate (Na-alginate)
Lead (II) tungstate/TEG nanoparticles (PbWO₄@TEG NPs) powder
abellaite/TEG nanoparticles (PbCO₃@TEG NPs) powder
Glucono-δ-lactone (GDL)

Procedure: 1.00 g of Na-Alginate was added to 50 mL DI water to prepare a 2% (w/v) Na-alginate solution. Approximately 5 g of the abellaite @TEG NPs powder and 5 g of the PbWO₄@TEG NPs powder were dispersed in 20 mL DI water in a 250 mL beaker. The solution was then sonicated for about 5 minutes to uniformly disperse the NPs powder in the solution. 25 mL of the 2% (w/v) Na-alginate solution was added to the abellaite @TEG NPs suspension and mixed thoroughly. 1.0 g of GDL was dissolved in 5 mL DI water. The 5.0 mL GDL solution was added to the 45 mL nanocomposite suspension to initiate crosslinking and mixed thoroughly. Strong acid or strong base may be added dropwise to adjust the pH to within the 5.5-6.5 range to optimize crosslinking for the most effective gelation. Gelling time (45-50 min) was recorded using a stopwatch. GDL, Na-alginate, and abellaite @TEG NPs concentrations may be varied to control the gelling time.

TABLE 3
Contrast Agent/Gel Precursor Solution Compositions

| Gel Component | % (w/w)^{a} | % (w/w)^{b} | % (w/w) Range^{c} |
|---|---|---|---|
| abellaite @TEG | 16 | 8 | 1-50 |
| PbWO₄@TEG | 0 | 8 | 0-50 |
| GDL | 1 | 2 | 0.01-5 |
| Na-alginate | 1 | 1 | 0.01-2 |
| Water | 82 | 81 | 40-99 |

^{a}Typical composition for contrast agent/gel precursor solution using abellaite nanoparticles only.
^{b}Typical composition for contrast agent/gel precursor solution using PbWO₄ nanoparticles.
^{c}Possible ranges for constituent materials.

### Micro-Ct X-Ray Imaging

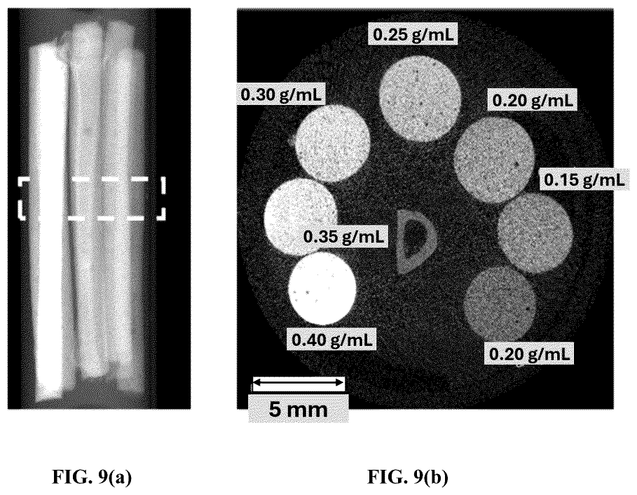

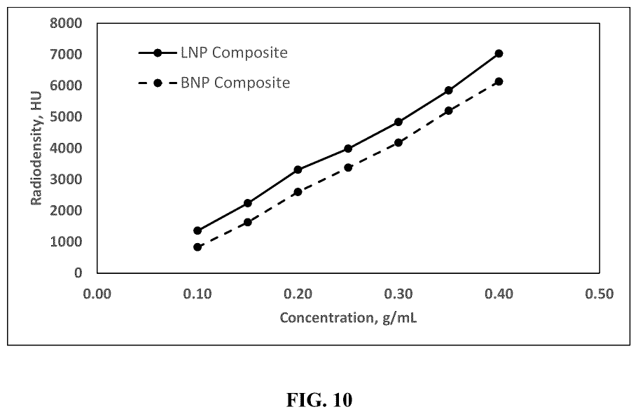

Gels with total LNP concentrations 0.10, 0.15, 0.20, 0.25, 0.30, 0.35, or 0.40 g/mL were prepared and cast in 4.30 mm diameter vinyl tubes. The tubes with containing gels were embedded into agarose with a formalin-fixed rat tibia for reference. The group of samples were scanned in a single scan shown in FIG. 9 with micro-CT (Micro-CT 35, ScanCo Medical, Bruttisellen, Switzerland; X-ray tube potential 70 kVp, integration time 300 ms, X-ray intensity 145 A, isotropic voxel size 10 m, frame averaging 1, projections 1000, and high-resolution scan). Micro-CT image intensity was converted to radiodensity measured in Hounsfield units (HU) using the formula HU=1000×(image intensity −image intensity of water)/(image intensity of water). Image intensity as a function of nanoparticle concentration is shown in FIG. 10 for both abellaite LNPs-GDL-alginate and (previously reported and patented) BaSO₄ BNPs-CaCO₃ CNPs-GDL-alginate nanocomposite gels.

### Example 2

### Synthesis of Abellaite-Containing Nanoparticles

### Materials

Lead (II) nitrate (PbNO₃ anhydrate, purity >99.0%), sodium carbonate (Na₂CO₃), potassium carbonate (K₂CO₃), cesium carbonate (Cs₂CO₃), ammonium carbonate ((NH₄)₂CO₃), ammonium bicarbonate (NH₄HCO₃), and sodium bicarbonate (NaHCO₃) were purchased from Sigma-Aldrich (St Louis, MO, USA). Tetraethylene glycol (TEG; 99%, Alfa Aesar, Thermo Fisher scientific, USA) was used as supplied.

### Synthesis of LNPs

The synthetic protocols for LNPs (lead-based nanoparticles) were based on a polyol arrested precipitation reaction between lead (II) nitrate and soluble carbonate solutions in a mixture of TEG and water. High-intensity sonication using a sonic dismembrator (US Solids 1200 W Ultrasonic Homogenizer) at a power of 900 W was applied during the nanoparticle formation.

A 1.45 M solution of lead (II) nitrate in water was prepared by dissolving 12 g of lead (II) nitrate in 25 mL of DI water. The amounts and concentrations of the carbonate precursor solutions in water for the six reactions studied are shown in Table 4. The Pb(NO₃)₂ solution was combined with 100 mL of TEG using a glass rod to promote full dissolution of the TEG. To this mixture was added the appropriate carbonate precursor solution dropwise with a Pasteur pipette over the course of 10-15 minutes, while being subjected to high-intensity sonication using the sonic dismembrator. The final TEG concentration after combination was 62% (v/v) for the reactions using the carbonate precursors and 38% to 48% (v/v) for the reactions using bicarbonate precursors. The sonic dismembrator was tuned so that it was highly pitched, and the solution was visibly mixed (this was readjusted throughout the addition). Drops of the corresponding carbonate precursor were added close to the sonic dismembrator probe at a rate of 2 drops per second. After the contents of the pipette were fully dispensed, the beaker was moved so that the probe reached the edges in a circular twisting motion and was then returned to the middle of the beaker. Once the addition was complete, a watch glass was placed on top of the beaker holding the suspended LNPs for 5-6 h. Finally, the LNP suspension was centrifuged 4 times (3500 rpm, 15 min) and washed with DI water between each centrifugation, then dried in an oven at 110° C. for 10-12 h to yield the product powder.

TABLE 4
The mass of carbonate precursor and volume of water used
to make the carbonate-containing solution, its resulting
molarity and pH, and the final concentration of TEG in
the combined reaction mixture using the solution.

| | Car- | | Carbonate | | | |
|---|---|---|---|---|---|---|
| | bonate | Water | Solution | Solu- | Final TEG | |
| Carbonate | Mass | Volume | Molarity | tion | Concentra- | Yield |
| Precursor | (g) | (mL) | (mol/L) | pH | tion (v/v) | (g) |
| Na₂CO₃ | 5.72 | 20 | 2.7 | 12.38 | 62% | 9.8 |
| K₂CO₃ | 7.46 | 20 | 2.7 | 12.38 | 62% | 9.1 |
| Cs₂CO₃ | 17.6 | 20 | 2.7 | 12.38 | 62% | 8.9 |
| (NH₄)₂CO₃ | 5.19 | 20 | 2.7 | 9.20 | 62% | 8.6 |
| NaHCO₃ | 9.06 | 96 | 1.1 | 8.34 | 38% | 7.9 |
| NH₄HCO₃ | 8.54 | 40 | 2.7 | 7.85 | 48% | 7.6 |

### Particle Diameter and Dispersibility

Dynamic light scattering (DLS) measurements were recorded using a Zetasizer Ultra (Malvern Panalytical Ltd., Worcestershire, UK), fitted with a 10 mW 632.8 nm helium-neon laser, using noninvasive backscatter with a scattering angle of 173° and the temperature at 25° C. Powder X-ray diffraction (PXRD) measurements were carried out with a Rigaku Miniflex 600 X-ray diffractor with Cu Kα radiation (40 kV, 15 mA). Crystalline phases were identified by comparison with the ICDD Crystallographic Database. The Scherrer equation, D=Kλ/β cos θ, was used to calculate the crystallite size of nanoparticles, where D is the particle diameter, λ is the wavelength of the X-rays, θ is the diffraction angle, β is the full-width-at-half-maximum, and K is a constant. The transmission electron microscopy (TEM) samples were prepared by suspending the dried nanoparticles in distilled water and then casting on Formvar TEM grids (Ted Pella, Inc., Redding, CA). Electron microscopy images were obtained with a JOEL 1200EX TEM instrument operated at 60 kV and a JOEL 1400 Plus (XR 80 Camera) TEM operated at HV=120 kV.

### Results and Discussion

Commercially available Pb(NO₃)₂ and carbonate-containing precursors were combined to produce a precipitate of NPs. Uncontrolled precipitation can result in the formation of large microscale particles. To control and limit the particle growth, capping agents were used to arrest the precipitation reactions and limit particles to the nanodimensional range. These ligands also increased the viscosity of the reaction medium which slowed the nanoparticle crystal growth. Additionally, high-intensity sonication was applied to prevent particle aggregation during the synthesis.

The polyol capping method is a well-established and reliable method for controlling the growth of nascent nanoparticles of both elements and compounds. In the present system, the capping agent bound to surface cations and passivated the surface, thus stabilizing the smaller nanoparticles. The capping agent, depending on molecular size, also increased the solution viscosity, to control (slow) the rate of the precipitation reaction. TEG provided a balance between nanoparticle surface coverage and solution viscosity.

PXRD analysis was used to identify the crystalline PbCO₃-containing phases through identification using the ICDD database. Scherrer peak-width-at-half-height analysis was used to determine the average TEG-capped NP diffracting crystallite size. The results for the 5 reactions are listed in Table 5. All the nanoparticles had diameters in the 10 nm to 30 nm range. There were three different crystal forms produced: cerussite (Pb(CO₃)), hydrocerussite (2PbCO₃—Pb(OH)₂), and abellaite (NaPb₂(CO₃)2(OH)). Sodium carbonate produced abellaite nanoparticles exclusively. Potassium carbonate produced exclusively hydrocerussite. The ammonium carbonates and sodium bicarbonate produced exclusively cerussite. Cesium carbonate produced mixtures of cerussite and hydrocerussite with poor reproducibility in regard to the ratio of the phases, and thus was not further characterized.

TABLE 5
The identity and average crystallite size of the TEG-capped NP diffracting crystallites
produced using the different carbonate materials as precursors.

| Carbonate Precursor | Na₂CO₃ | K₂CO₃ | (NH₄)₂CO₃ | NaHCO₃ | NH₄HCO₃ |
|---|---|---|---|---|---|
| Crystallite Size (nm) | 21.2 ± 1.5 | 21.4 ± 3.3 | 13.1 ± 4.0 | 24.6 ± 2.0 | 18.7 ± 7.3 |
| Product Identity | Abellaite | Hydrocerussite | Cerussite | Cerussite | Cerussite |

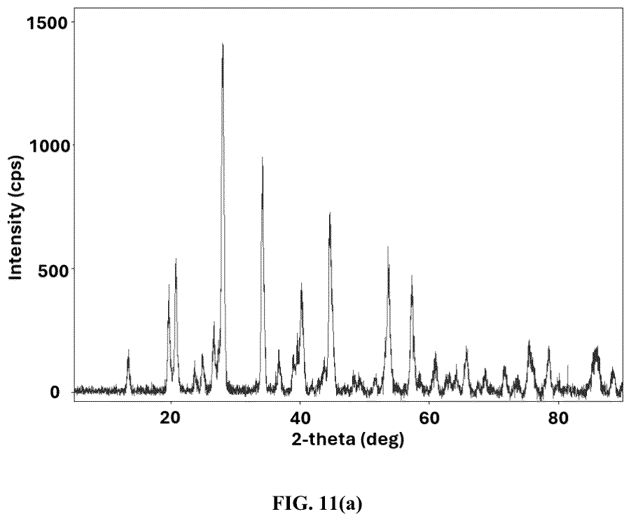

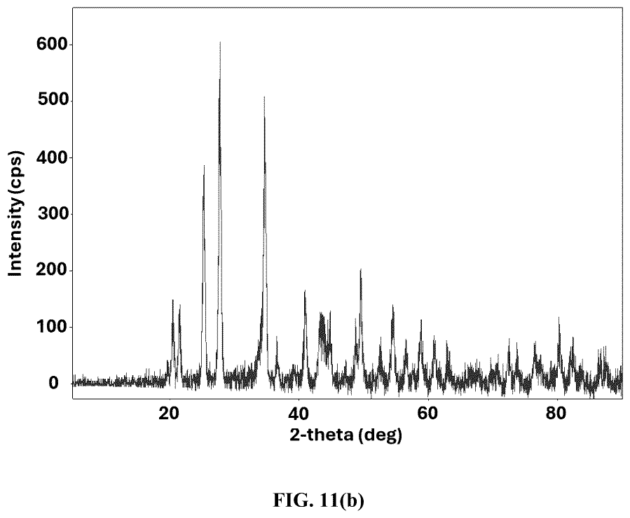

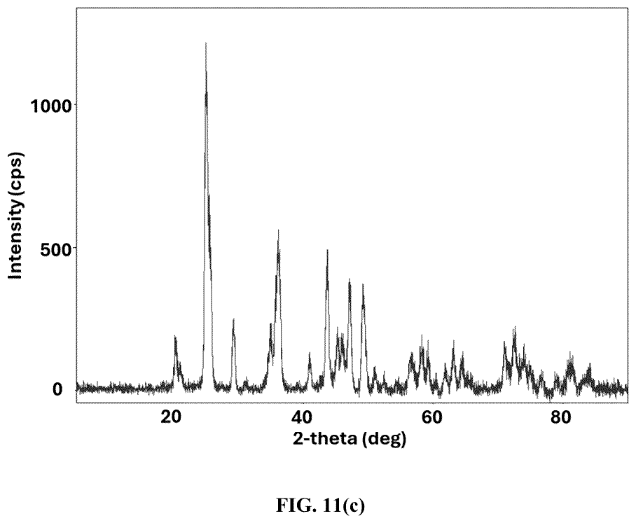

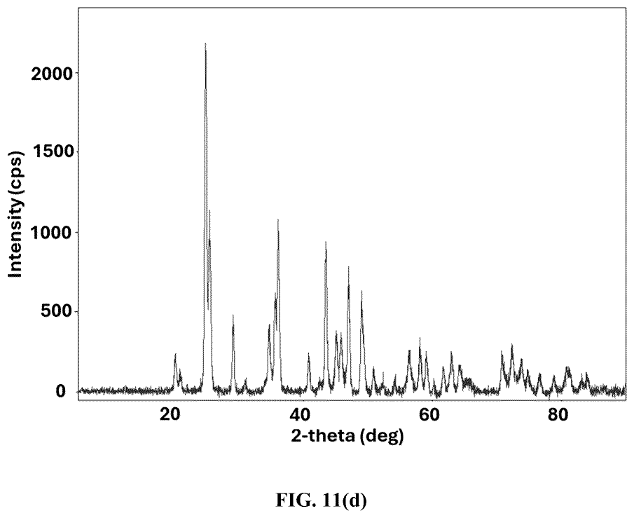

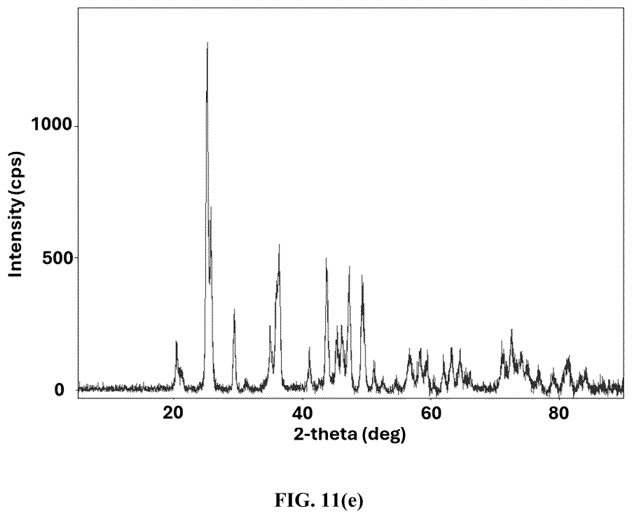

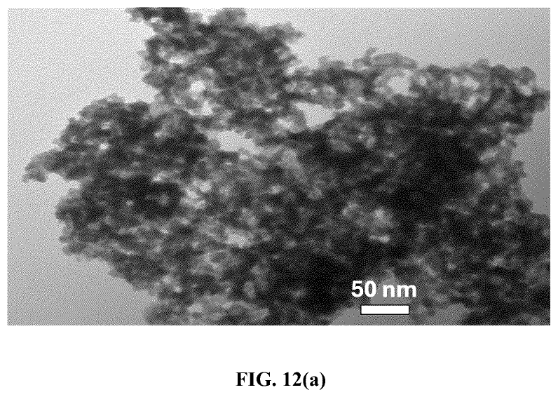

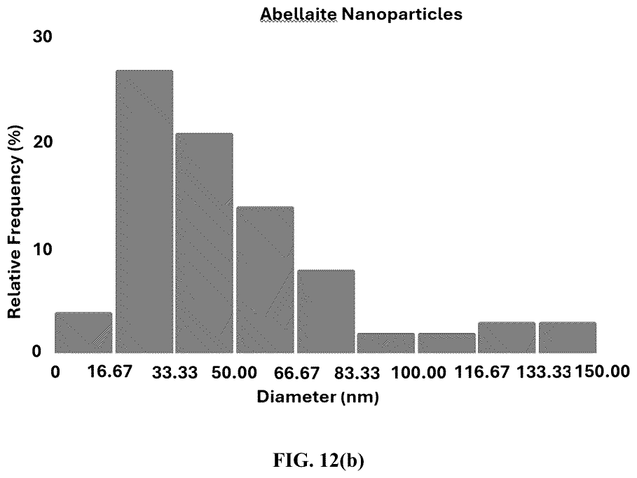

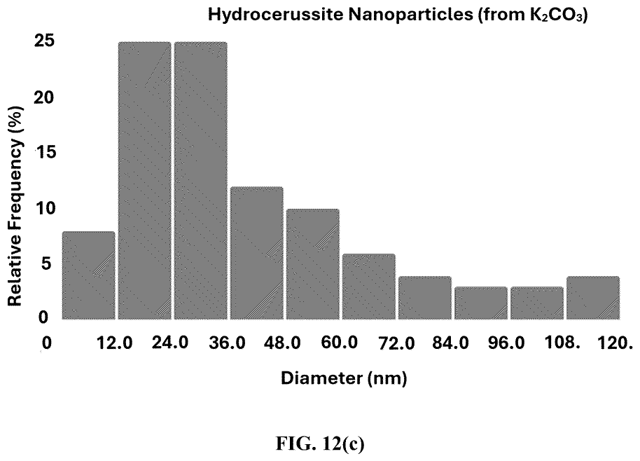

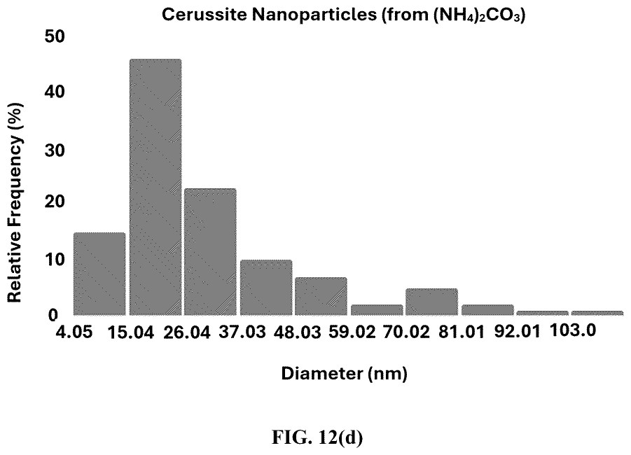

PXRD analyses of the products for the reactions of the five carbonate precursors are shown in FIGS. 11(a)-11(e). There are multiple known lead (II) carbonate phases with cerussite and hydrocerussite being the most commonly observed. Cerussite nanoparticles have previously been reported. A hydrocerussite nanophase has been reported but these were 2D materials with 2 dimensions on the micron scale. The inventors are unaware of any previous reports of GD hydrocerussite nanoparticles. The abellaite phase is much less common. The X-ray diffraction pattern for the abellaite phase was first observed in 1982 by Brooker and coworkers. The structure of abellaite was correctly characterized as NaPb₂(CO₃)₂(OH) in 2002 by Belokoneva and coworkers. Its existence in nature was first reported in 2017 by Ibañez-Insa and coworkers. For abellaite, no nanoparticle phases have previously been reported.

The reactions to produce each of the three crystal phases are shown in reactions (A)-(C). The carbonate reactant is listed as HCO₃⁻ for reaction (B) as this was the predominant form of the ion at the pH for the ammonium carbonates and sodium bicarbonate solutions. For reactions (A) and (C) the reactant is listed as CO₃²⁻ as this was the predominant form of carbonate at the pH of the metal carbonate solutions. The calculated pH values for the six reactants are shown in Table 4.

2Pb(NO₃)₂(aq)+Na⁺(aq)+2CO₃²⁻(aq)+OH⁻(aq)→NaPb₂(CO₃)₂(OH)(s)+4NO₃⁻(aq) (A)

Pb(NO₃)₂(aq)+HCO₃⁻(aq)→PbCO₃(s)+2NO₃⁻(aq)+H⁺(aq) (B)

3Pb(NO₃)₂(aq)+2CO₃²⁻(aq)+2OH⁻(aq)→2PbCO₃⁻Pb(OH)₂(s)+6NO₃⁻(aq) (C)

For comparison, the precipitation reaction of sodium carbonate with lead (II) nitrate was carried out in pure water (no TEG) without sonication. The formation of a mixed-phase product consisting of cerussite and abellaite was observed. The formation of hydrocerussite, a hydroxide-containing phase, is known to be preferred over that of cerussite in water at higher temperatures. Previous reports on abellaite have utilized hydrothermal synthesis methods. These results were consistent with the preferred formation of abellaite, also a hydroxide-containing phase, with energy input from sonication or microwaves.

Additionally, the abellaite and hydrocerussite phases were formed by the more alkaline reactants. This is consistent with the need for OH⁻ reactant for these phases. The more neutral reactants, ammonium carbonates and sodium bicarbonate, produced the cerussite phase. This is the traditionally predicted phase based on standard solubility rules. Thus, the presence of hydroxide in the solution drives the products through pathways (A) and (C).

The sizes, shape, and morphologies of the abellaite NPs were visualized using TEM and the particle diameters of 100 particles were determined from their respective TEM images. As previously observed in Example 1, NPs were mostly spherical in shape, with some agglomeration observed being most likely due to the TEM grid preparation method (See, FIG. 2(a)). The particle size distribution and the average particle size was determined to be 40±10 nm, consistent with results obtained in Example 1 (see, FIG. 2(b)).

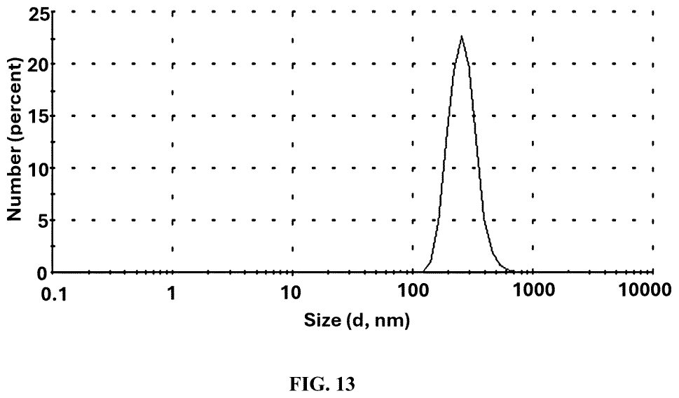

More detailed DLS analysis permits judgment regarding the colloidal dispersibility and homogeneity of LNPs, something that is important for potential capillary-micro-CT applications of these nanoparticles. Nanoparticles that aggregate quickly and easily fall out of suspension will resist uniform transportation through capillaries and be more likely to rupture blood vessels during perfusion. DLS size plots for the abellaite particles dispersed in liquid TEG are shown in FIG. 13. Qualitatively, the NPs were found to be well dispersed in liquid TEG. DLS sizing analysis revealed that NP colloidal dispersions were homogenous with monomodal size distributions. The nanoparticle sizes based on the DLS measurements are listed in Table 6. The TEM measurements for abellaite are in agreement with the DLS measurements, indicating that individual TEG-capped abellaite NPs do in fact disperse completely in aqueous media. The discrepancy with PXRD crystallite diameters for abellaite (25 nm) may be attributed to a combination of the nature of the core-shell nanoparticles, with an amorphous capping layer of TEG and NaPb₂(CO₃)₂OH as well as a small degree polycrystallinity within the nanoparticles. Abellaite NPs had measured zeta potentials of −56.8 nm±10.9 nm. This can be attributed to an exterior nanoparticle surface coating by excess CO₃²⁻ ions in the LNPs.

TABLE 6
The average zeta potential of the LNPs as determined using DLS measurements.

| Product | Abellaite | Hydrocerussite | Cerussite | Cerussite | Cerussite |
|---|---|---|---|---|---|
| Carbonate | Na₂CO₃ | K₂CO₃ | (NH₄)₂CO₃ | NaHCO₃ | NH₄HCO₃ |
| Precursor | | | | | |
| Zeta Potential | −56.8 ± 10.9 | −55.9 ± 8.8 | −24.1 ± 6.4 | −18.4 ± 6.0 | −18.0 ± 4.3 |
| (mV) | | | | | |

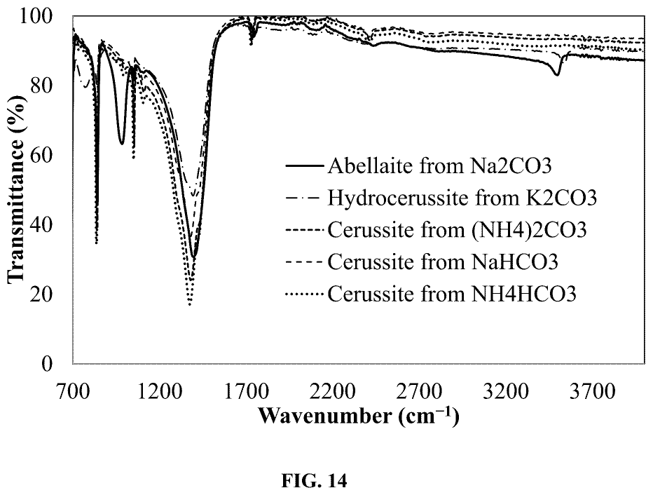

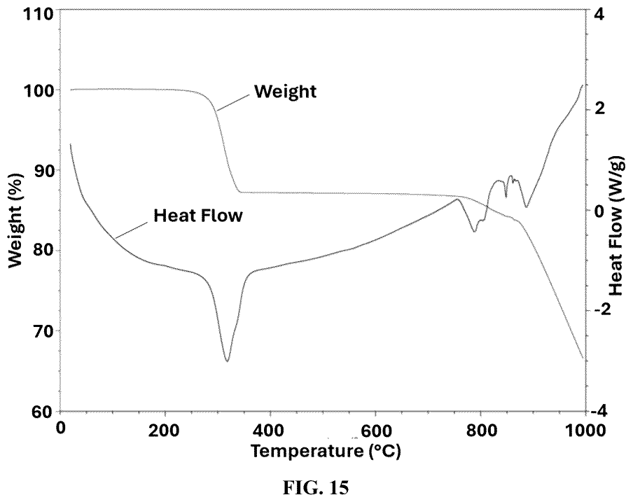

ATR-FTIR spectra for all three phases are shown in FIG. 14. The spectra for the products formed from (NH₄)₂CO₃, NaHCO₃, and NH₄HCO₃ all confirm cerussite as the primary product with absorption peaks at identical positions at 1730 cm⁻¹, 1390 cm⁻¹, 1110 cm⁻¹, 1050 cm⁻¹, and 835 cm⁻¹. The most intense absorption at 1390 cm⁻¹ attributed to the CO₃²⁻ υ₃ mode also bears a shoulder at 1420 cm⁻¹, not observed in the other carbonate-containing nanoparticles. The hydrocerussite material synthesized from K₂CO₃ shows similar absorptions with two notable additions at 3540 cm⁻¹ and 775 cm⁻¹ due to OH stretching and libration, respectively. Hydroxide ions are also clearly observable for the abellaite LNPs at 3500 cm⁻¹ and 988 cm⁻¹. The abellaite spectrum shows the same characteristic CO₃²⁻ absorptions as the other carbonate-containing phases (1737 cm⁻¹ and 1405 cm⁻¹, 1120 cm⁻¹, 1058 cm¹, and 842 cm⁻¹). In all spectra, there is evidence of very small quantities of TEG, the strongest absorptions for which normally appear at 3300-3400 cm⁻¹ (OH stretch) and 2800 cm⁻¹ (CH stretch), but these are extremely weak in the LNP spectra, likely due to the minimal surface coverage by very small quantities in comparison to the crystalline salt materials.

### Conclusions

This Example demonstrates the first synthesis and observation of abellaite nanoparticles of any dimension and of 0-D nanoparticles of hydrocerussite. The syntheses were performed using high power ultrasound in tetraethylene glycol (TEG). More alkaline carbonate precursor solutions lead to production of the hydroxide-containing phases (abellaite and hydrocerussite), while the more acidic precursor carbonates lead to production of the cerussite phase. Nanoparticle sizes were in the 10 nm to 40 nm range with roughly spherical particle shape. The hydroxide-containing phases are particularly useful in formation of gels for vascular CT imaging as the divalent metal cations can be released with lower production of the CO₂ gaseous product, which can lead to bubble formation within the gels.

### Example 3

NaPb₂(CO₃)₂OH Nanoparticle-Alginate Composite

### Materials

Lead (II) nitrate (PbNO₃; anhydrate, purity ≥99.0%), sodium carbonate (Na₂CO₃), alginic acid sodium salt [low viscosity, 4-12 cP, 1% in H₂O (25° C.)], poly(ethylene glycol) (MW 10,000 g/mol by GPC), poly(sodium 4-styrenesulfonate) (PSS, MW 70,000 g/mol by GPC), poly(acrylic acid) sodium salt (MW 5100 g/mol by GPC), and d-(+)-gluconic acid δ-lactone (GDL; C₆H₁₀O₆) were purchased from Sigma-Aldrich (St Louis, MO, USA), tetraethylene glycol (TEG; 99%, Alfa Aesar, Thermo Fisher scientific, USA), and polyvinyl alcohol (PVA, MW 30,000 g/mol, Merck KGaA, Germany) were used as supplied. Stock aqueous solutions of 2.0% w/v alginate, used in most experiments, were made by using alginic acid sodium salt powder with deionized (DI) water. After complete dissolution, the aqueous alginate was stored at 4° C. and was used within 1 week of mixing.

### Abellaite NP Synthesis

Abellaite NPs were synthesized using an arrested precipitation of NaPb₂(CO₃)₂OH. In 27 mL of DI water, 10 g of Na₂CO₃ was dissolved to make 2.61 M Na₂CO₃. Separately, 16 g of Pb(NO₃)₂ was dissolved in 100 mL of DI water to make 0.61 M Pb(NO₃)₂. The Pb(NO₃)₂ solution was combined with 100 mL of TEG using a glass rod to promote full dissolution of the TEG. To this mixture was added the Na₂CO₃ solution dropwise with a Pasteur pipette over the course of 10-15 min, while being subjected to high-intensity sonication using a sonic dismembrator (US Solids 1500 W Ultrasonic Homogenizer). The final TEG concentration after combination was 44% (v/v). The LNP solution was centrifuged (3500 rpm, 15 min), and the solid was washed and dried in an oven at 100-150° C. for 10-12 h to yield 10 g of dry white powder.

### Microwave Irradiation Synthesis

As an alternative method for preparing abellaite NPs by sonic dismembrator engergization, a microwave irradiation synthesis method was developled. The Pb(NO₃)₂ solution was combined with 100 mL of TEG using a glass rod to promote full dissolution of the TEG. To this mixture was added the appropriate carbonate precursor solution dropwise with a Pasteur pipette over the course of 10-15 minutes, while being stirred in a microwave reactor (900 W power output with probe temperature control set to 80° C.). The final TEG concentration after combination was 62% (v/v) for the reactions using the carbonate precursors and 38% to 48% (v/v) for the reactions using bicarbonate precursors. The reaction mixture was stirred with continuous microwave irradiation until all of the Pb(NO₃)₂ solution was added. Once the addition was complete, the mixture was removed from the microwave reactor and a watch glass was placed on top of the beaker holding the suspended LNPs for 5-6 h. Finally, the LNP suspension was centrifuged 4 times (3500 rpm, 15 min) and washed with DI water between each centrifugation, then dried in an oven at 110° C. for 10-12 h to yield the product powder.

### Alginate Hydrogel Preparation

To prepare a gel cylinder for gelation time and viscosity analysis, 0.20 g of alginic acid sodium salt was added to 5 mL of DI water. Approximately 1.0, 1.5, 2.0, 2.5, 3.0, 3.5, and 4.0 g of the LNP powder was dispersed in 5 mL of DI water to prepare various LNP concentrations of 0.10, 0.15, 0.20, 0.25, 0.30, 0.35, and 0.40 g/mL. The LNP solution was then sonicated for 5 min to disperse the NP powders in the solution. Next, the alginic acid sodium salt solutions and LNP suspension were combined. Finally, 0.052 g of GDL was added to the combined mixture and dissolved to initiate the cross-linking.

### Particle Diameter and Dispersibility

Transmission electron microscopy (TEM) samples were prepared by suspending the dried nanoparticles in distilled water and then casting on Formvar TEM grids (Ted Pella). Electron microscopy images were obtained with a JOEL 1200EX TEM instrument operated at 60 kV and a JOEL 1400 Plus (XR 80 Camera) TEM operated at HV=120 kV.

### Gelation Time and Viscosity Measurement

The gelation of alginate hydrogel was studied using the inverted test tube method. The gelation time was determined when the alginate did not flow at the point of inversion during gelation, which was executed every 30 s at room temperature and measured using a stopwatch. Different GDL, alginate, and LNP compositions were tested to obtain the optimal gelation time. To determine the relative storage modulus and working times of the hydrogel composites, rheometric measurements were taken on an AR 2000ex rheometer (TA Instruments, New Castle, DE). Time sweeps were performed using a flat, parallel plate geometry and a constant 2% strain, angular frequency of 1 Hz, and temperature of 37° C.

### Hydrogel Compressive Properties

To determine if solidified gels could withstand postmortem tissue harvesting and other handling, compressive testing was performed. Cylindrical gel samples (21 cm diameter and 10 cm height) were prepared for gelatin and LNPs-alginate formulations and compared with commercially available Microfil samples (MV-122, Flow Tech, Inc., Carver, MA). Specimen thickness was estimated using a Mitutoyo IP54 digital micrometer (Mitutoyo American Corp., Aurora, IL). Then, the gels samples were compressed at an extension rate of 10.0 mm/mm to 50% strain (MTS Criterion, Eden Prairie, MN, 100 N load cell). Peak load was recorded in grams-force using Test Suite Elite software.

### Radiodensity

Gels with LNP concentrations 0.10, 0.15, 0.20, 0.25, 0.30, 0.35, or 0.40 g/mL were prepared and cast in 4.30 mm diameter vinyl tubes. The tube-filled gels were embedded into agarose with a formalin-fixed rat tibia for reference. The group of samples were scanned in a single scan with micro-CT (Micro-CT 35, ScanCo Medical, Brüttisellen, Switzerland; X-ray tube potential 70 kVp, integration time 300 ms, X-ray intensity 145 A, isotropic voxel size 10 m, frame averaging 1, projections 1000, and high-resolution scan). Micro-CT image intensity was converted to radiodensity measured in Hounsfield units (HU) using the formula HU=1000×(image intensity−image intensity of water)/(image intensity of water).

### Computational Methods

All calculations were performed using ORCA version 5.0.1 on a CentOS Linux 7 cluster employing Open MPI version 4.0.5. All of the calculated interaction energies were BSSE corrected using the counterpoise method of Boys and Bernardi. Chemcraft was used to construct all starting structures and to visualize the computational results. Starting geometries were assembled by arranging strands of alginate polymer and metal ions to approximate egg-crate structures. Alginate polymer strands were created by attaching monomer units to achieve the desired strand length and then terminated on each end with hydrogen atoms. Alginate polymers are commercially available with differing distributions of G- and M-residues; this example used exclusively G-residues and assumed that the results would be similar for other residue distributions. Atoms counts (357 atoms for a hexamer unit) made it necessary to use computational methods appropriate for large systems. The Grimme HF-3c and PBEh-3c methods in ORCA 5.0.1 were tested for this purpose. HF-3c gave more stable and reliable geometry, and was used to determine all of the values in this example.

Example 1 describes a method for synthesis of nanocrystalline NaPb₂(CO₃)₂OH phases in TEG-capped NPs. The particles were characterized through Powder X-ray diffraction and Dynamic Light Scattering, with the average TEG-capped NP diffracting crystallite size observed to be 25±5 nm. The NP particles were mostly spherical in shape, with some agglomeration observed being most likely due to the TEM grid preparation method. The average particle size (inorganic core plus organic cap) was determined to be 40±10 nm. This result was consistent with prior DLS sizing analysis that revealed that LNP colloidal dispersions were homogenous with monomodal size distributions with nanoparticle diameters of 40±5. LNPs had measured zeta potentials of −39 mV, attributed to an exterior nanoparticle surface coating by excess CO₃²⁻ ions in the LNPs.

### Production and Mechanical Properties of Abellaite NP-Alginate Nanocomposite Gels

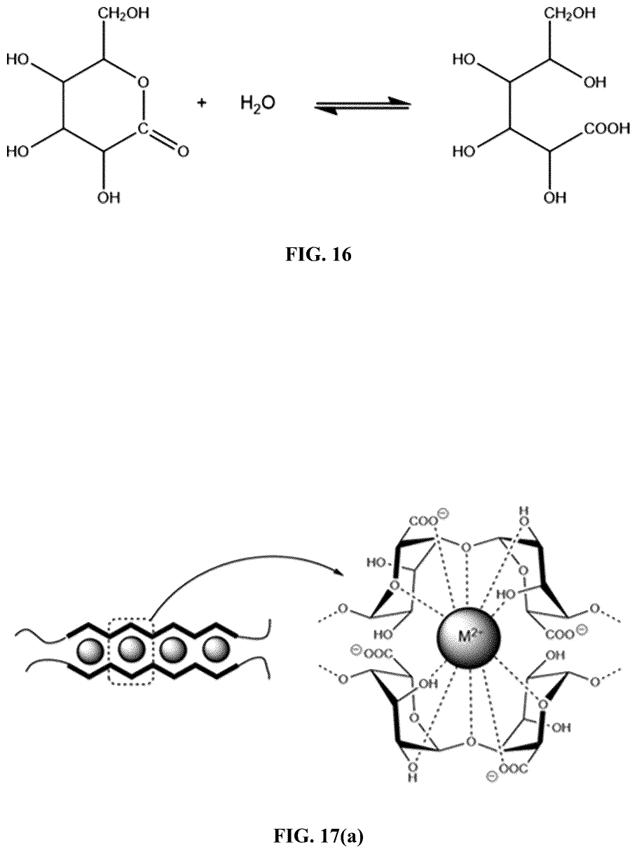

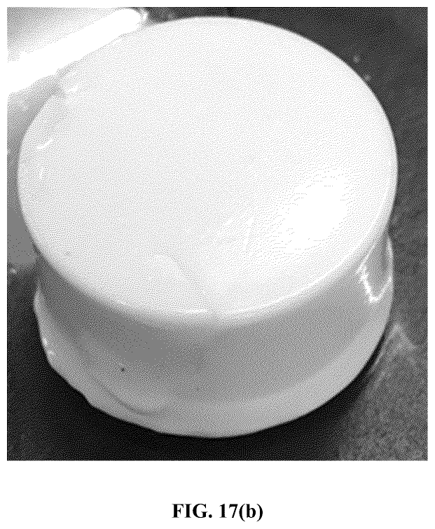

As a prelude to preparing nanocomposite gel precursor suspensions for capillary perfusion experiments, we developed a synthetic protocol to cast nanocomposite gel cylinders for the purpose of probing the gelling suspension for viscosity and gelling time as well as to examine the mechanical properties of the final cast gel. Alginate was selected, following the results of our previous BNP work. The precursor liquid nanocomposites were prepared by mixing abellaite NPs and GDL with a solution of 2% alginic acid sodium salt (2% w/v). GDL is converted to gluconic acid in the presence of water (FIG. 16), which slowly lowers the pH of the solution. In the acidic condition, NaPb₂(CO₃)₂OH in the abellaite NPs dissociates, releasing Pb²+ and CO₃²⁻ ions into the solution. The free Pb²⁺ cations cross-link with the alginate polymer, slowly transforming the liquid nanocomposite into a solid gel. This eliminates the need for additional CaCO₃ NPs in the final nanocomposite. The cross-linking effects a more rigid long-range order according to the so-called ‘egg-box’ model of divalent cation binding with the alginate hexose rings (FIG. 17(a)). The resulting gel cylinders are shown in FIG. 17(b).

To analyze gelation time to form a structurally stable, but flexible, solid gel that will avoid leakage of the nanocomposite components before or during micro-CT analysis, compositions of abellaite NPs, GDL, and alginate (the primary components that determine the gelation rate) were varied, and the gel formation was studied using the inverted test tube method. The gelation times for an array of LNP/GDL/alginate compositions was 30-40 minutes.

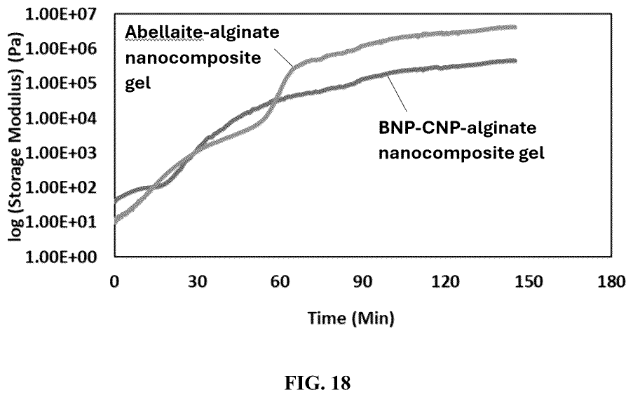

Rheological assessment of storage modulus versus time was used to gauge viscosity during the gelation process. Results are presented in FIG. 18. Storage moduli of pure water and the ungelled alginate solution remained constant and low (<50 Pa). Inclusion of the BNPs, CNPs and GDL, resulted in a peak storage modulus of ca. 4.5×10⁵ Pa after ca. 2 hours of mixing. On the other hand, inclusion of abellaite NPs and GDL in the gel preparation protocol resulted in the storage modulus plateauing after ca. 2 hours at ca. 4.3×10⁶ Pa. The storage modulus increased to ca. 10³ Pa within 30 minuntes gelation time. Thus, a working gelation window of 30-35 minutes after mixing was determined for this composition.

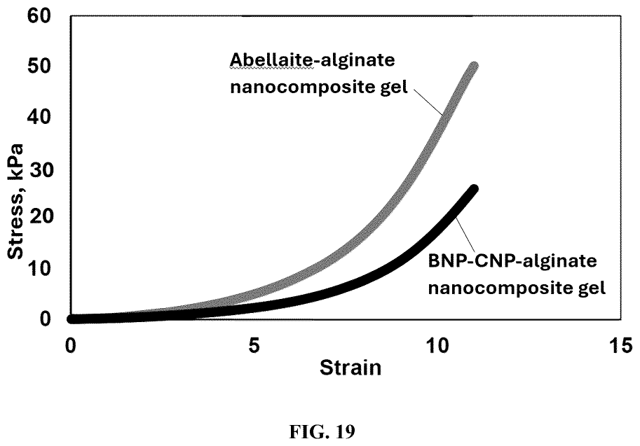

Compressive stiffness is another important parameter for the intended micro-CT application because after transforming a liquid composite into a solid gel, the vessel cast must be adequately robust to withstand tissue harvesting and handling without leaching from the vasculature or significantly altering vessel size and shape. The abellaite NP-alginate gel showed greater stiffness, peak stress was recorded at 55 kPa. This was stiffer than the BNPs and was a marked improvement over the Microfil and BNP composite (FIG. 19).

Solidified 12% commercial gelatin provides an ideal mechanical stiffness for a solid gel scaffold with entrapped nanoparticles intended for micro-CT imaging, and with a peak stress value of 91 kPa. However, gelatin has a significant disadvantage, in that it must be forced into solution by heating and remain viscous enough for injection, which is undesirable for micro-CT applications. The high temperature required during injection may cause tissue damage, rendering the sample useless for further experiments like histological analysis. The current industry standard for micro-CT analysis is Microfil, a silicon-based composite material. The mechanical stiffness of the Microfil is the lowest of the four samples studied (Table 7) with a peak stress value of just 12 kPa.

TABLE 7
Stress Testing of Gelatin, Microfil, BNP Calcium Carbonate
Alginate, and Calcium Carbonate Alginate Gel Cylinders

| Sample | Peak Stress, kPa | Elastic Modulus, kPa |
|---|---|---|
| Abellaite NP-alginate gel | 55 ± 5 | 318 ± 4 |
| BNP-alginate gel | 25 ± 1 | 112 ± 2 |
| 12% gelatin | 91 ± 5 | 604 ± 5 |
| Microfil | 12 ± 1 | 60 ± 1 |

All cylinders of diameter 21 cm and height 10 cm.

### Computational Study of the Metal Alginate Complex

A series of structure and energy calculations were performed on various model metal dication-alginate compositions to analyze the relative structural stabilities of the gels. All calculations were performed using the DFT method in ORCA 5.0 program package, def2-TZVP and def2-SVP basis sets were used for geometry optimizations. The terms “monometal”, “dimetal”, “trimetal”, and “hexametal” refer to the number of M²⁺ and alginate ions in the structure. The appropriate number of alginate monomer units were included in order to build the egg-crate structure.

The atomic coordinates of the metal-alginate species are not known, and the real species is likely to contain many atoms. Both of these are impediments to computational analysis and require that a computation study develop smaller model species that accurately represent the interactions in the real system.

The initial model for the computational study was the monometal system. The calculations showed the binding energy for Ca²⁺ was greater than for Pb²⁺, with the difference in binding energies being 0.167 Hartree=438.7 kJ/mole=285 kcal/mole (Table 8). These binding energy values implied that Ca²⁺ binding to alginate was stronger than for Pb²⁺ indicating that the Ca²⁺ cross-linked structures should be stiffer and stronger than the Pb²⁺ cross-linked structures, which was inconsistent with our viscosity and other experimental measurements.

TABLE 8
Binding energy for monometal systems.

| Cations | Binding Energy |
|---|---|
| Ca²⁺ monometal | −1519.8 kJ/mole |
| Pb²⁺ monometal | −1081.1 kJ/mole |

Calculations with bimetallic systems also gave results that are not consistent with the experiments. Visual comparison of the monomer and dimer systems to the egg-crate model showed that the alginate units have more freedom of motion to encapsulate the metal ions in monomer and dimer units than is possible in the full alginate polymer. As a result, the binding energies in these smaller systems may not be representative of the binding energies in the actual M²⁺-alginate gel systems. To determine binding energies for systems that are more representative of the egg-crate model, the sizes of the model systems were increased to trimers and hexamers.

The large numbers of atoms in the trimetal and hexametal systems make these difficult and time-consuming to calculate optimized structures. Several years ago, Grimme reported (and implemented in a recent Orca update with designation HF-3c) a semi-empirical method for large systems like RNA, DNA, proteins, and polymers. (See, Bannwarth et al., WIREs Computational Molecular Science, 2021, 11 (2), e1493). It involves a rather small basis set and very limited electron correlation which makes it go faster, and it includes empirical dispersion corrections (which are fast) to compensate for the other limitations. This method was used to study trimetal and hexametal systems.

The HF-3c calculations for the trimetal system showed the binding energy for Pb²⁺ is greater than for Ca²⁺. The difference in binding energies is 0.453 Hartree=1178 kJ/mole=285 kcal/mole (95 kcal/mol-monomer unit) (Table 10). The results for the hexametal show the Pb²⁺ binding energy to be larger than that for Ca²⁺ by 0.1794 Hartree=471.2191 kJ/mole=112.6242 kcal/mole (90 kcal/mol-monomer unit) (Table 11).

TABLE 10
Binding energy for trimetal systems

| Cations | Binding Energy |
|---|---|
| Ca²⁺ trimetal | −8183.8 kJ/mole |
| Pb²⁺ trimetal | −10062.1 kJ/mole |

TABLE 11
Binding energy for hexametal systems

| Cations | Binding Energy |
|---|---|
| Ca²⁺ hexamer | −16710.3307 kJ/mole |
| Pb²⁺ hexamer | −17181.5498 kJ/mole |

Having obtained monometal and hexametal structures for both lead and calcium, the flexibility of alginate monomer units and hexametal units was analyzed and determined that the monometal binding energies were not representative of the experimental conditions. When the Ca and Pb monometal units are superimposed, the root-mean-square deviation (rmsd) for alignment of atoms was 2.23 A, and for the hexametal it was 1.53 A. As the size of the alginate unit increased and the degree of crosslinking increased, it was expected that the rmsd value would become smaller and the binding energy would depend more on the metal-alginate interaction and less on the flexibility of the alginate to deform as it surrounds the metal cation.

### Radiodensity of Nanocomposite Gels

Nanocomposite radiodensity will be critical for distinguishing the vascular networks into which they have been injected, especially in volumes that contain radiodense tissues like the bone. As the concentration of heavily X-ray scattering elements such as lead increases, so should the radiodensity of the nanocomposite. Gels with seven distinct LNP concentrations ranging from 0.10 to 0.40 g/mL were prepared and cast in vinyl tubes. The samples were collectively embedded into agarose along with a formalin-fixed rat tibia for reference and scanned by micro-CT to determine their radiodensity and establish the minimum concentration needed for differentiation from bone. The progressive increase in radiodensity is evident, and in comparison, with the embedded rat tibia, the highest concentration of abellaite NPs studied (0.40 g/mL) clearly yields a much brighter image. The measured X-ray intensity may be converted to radiodensity HU and plotted versus abellaite NP concentration in the gels, affording a clearly linear relationship. However, as storage modulus of the injected gelling solution also increases with abellaite NP concentration in the suspension, it is also incumbent upon us to identify the minimal abellaite NP loading needed for adequate imaging.

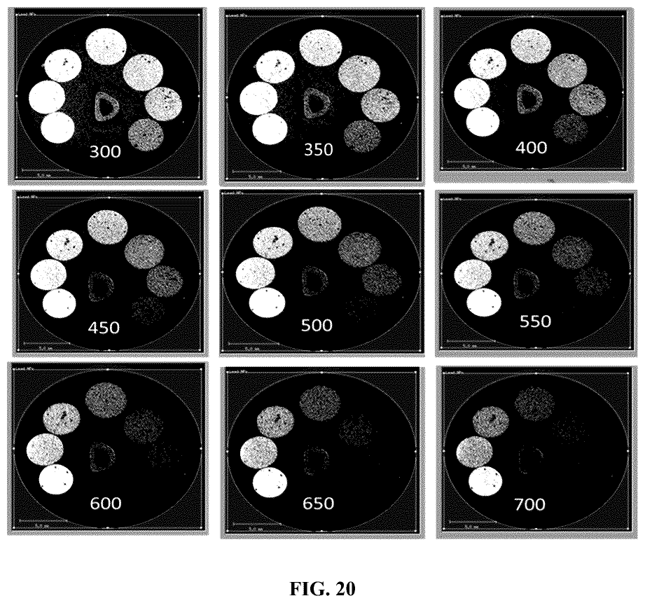

The images in FIG. 20 indicated that the minimum abellaite NP loading should thus be greater than 0.20 g/mL (3312 HU) because the radiodensity for this sample was roughly the same as that as for the rat tibia (3158 HU) and higher than that for Microfil (750 HU) at the same X-ray intensity threshold. Several bright spots were noted in the images and can be attributed to some patchy agglomeration within the gel structure once set. This did not appear to be a function of the age or shelf-life of the nanoparticles, as they were stable and did not degrade (as determined by PXRD analysis of nanoparticles that were several months old).

Abellaite (NaPb₂(CO₃)₂OH) nanoparticles were synthesized and incorporated into alginate gels for applications in postmortem micro-CT vascular network imaging. The final LNP-alginate nanocomposite was determined to control gelation time to within a 30-35-minute window. The stiffness of the developed nanocomposite gel was improved over that of commercially available Microfil and previously reported BNPs. The abellaite NP system of the present disclosure has an advantage over BNPs in that additional CNPs within the nanocomposite are not necessary to initiate cross linking. This reduces the solids loading within the composite and should provide better flow within the vasculature. Finally, micro-CT analysis determined that the minimal nanocomposite radiodensity for studying the vasculature in the vicinity of bone could be achieved with an LNP concentration of 0.20 g/mL in the injected gelling suspension. This lower limit of detection implies that the LNPs have higher intrinsic X-ray contrast than do the BNPs. This allows a reduced solids loading in the gel for LNPs relative to BNPs.

### Example 3

Synthesis and Gelation Chemistry of Ultrasmall (Sub-5 nm) Bi₂O₃ Nanoparticles

### Materials

Bismuth(III) nitrate pentahydrate (BiN₃O₉; ≥98.0%), sodium hydroxide (NaOH; >97.0%), tetraethylene glycol (TEG; 99%), and d-(+)-gluconic acid δ-lactone (GDL; C₆H₁₀O₆) were purchased from Sigma-Aldrich. Alginic acid, sodium salt [350 to 550 mPa·s, 1% in H₂O (20° C.)] was purchased from Acros Organics.

### Synthesis of Bismuth Nanoparticles (BiNPs)

2.5 grams of bismuth(III) nitrate pentahydrate were added to 25 mL of TEG. This mixture was then subjected to high intensity sonication (900 W) using a sonic dismembrator (US Solids 1200 W Ultrasonic Homogenizer) while being continuously stirred using a magnetic stir bar. During sonication, 10 mL of deionized water was added dropwise over the course of 5 minutes, followed by the dropwise addition of 16 mL of 1M sodium hydroxide solution over the course of 5 minutes. The final TEG concentration after combination was 49% (v/v). The BiNP solution was then centrifuged (3000 rpm, 15 min) and washed with DI water multiple times, then dried in an oven at 110° C. for 10-12 hours to yield about 1.1 grams of dry white powder.

### Preparation of Alginate Hydrogels

0.1 g of alginic acid, sodium salt was dissolved in 10 mL of DI water to form a 1% alginate solution. 2 g of the dry BiNP powder was added to 10 mL of DI water and then dispersed by 1 minute of high intensity sonication (900 W) using a sonic dismembrator. The alginate solution and the BiNP suspension were combined to form a solution of 0.1 g/mL BiNPs in 0.5% alginate. Finally, 0.224 g of GDL was added to the combined mixture and dissolved to initiate cross-linking. This method caused a stiff gel to fully form in about 30 minutes.

### Results and Discussion

Powder X-ray diffraction (PXRD) was used to characterize the bismuth-containing phase through identification using the ICDD database. Scherrer peak-width-at-half-height analysis was used to determine the average TEG-capped NP diffracting crystallite size. The resulting diffraction pattern identified the NPs as bismuth(III) oxide and the size is in the 2 nm range (±0.5 nm).

This Example provides a simple method to make very small (crystallites in the range of 2 nm) Bi₂O₃ nanoparticles. Synthesis or production of very small (sub-5 nm) Bi₂O₃ nanoparticles have not been reported by any groups. These are the smallest GD nanoparticles of this composition known to the inventors. There are two reports of non-oxide bismuth-containing nanoparticles: one of metallic bismuth NPs that are 5 nm and one of bismuth ferrite NPs that have 3-16 nm diameters.

Advantageously, the method of the present disclosure does not require strong acids as required in other methods that produce larger (−20-50+ nm) NPs. While the method of the present disclosure uses strong base, the base is only used in stoichiometric proportions and not as a solvent system.

The resultant Bi₂O₃ nanoparticles are also very stable. The Bi₂O₃ nanoparticles react with GDL in the presence of alginate to make stable hydrogels. This process does not lead to the production of gases (like CO₂ from the carbonates) which minimizes the chances for bubble formation. The presence of GDL as an activator for gel formation is quite unusual for in the method of the present disclosure. Bi₂O₃ is typically only soluble at extremely low pH values around 0. GDL does not lower the pH to that level. The extremely small size of the Bi₂O₃ NPs could provide a very high surface energy that allows release of Bi³⁺ at modest pH values. Bismuth-containing nanoparticles have many applications including biomedical applications. Bismuth is useful as the largest atomic mass nucleus that is “effectively” stable (lifetime>>estimated age of the universe). Bismuth and its compounds are generally non-toxic (with exceptions), which is unusual for the heavy metals in this portion of the periodic table. This high mass makes it a very useful element for X-ray imaging methods.

The present disclosure provides a contrast agent embedded in an injectable polymeric matrix that can be delivered with a small-gauge needle that is minimally invasive. A novel nanoparticle structure is provided that effectively reacts with aqueous alginate solutions to produce stable gels and function as a contrast agent that is particularly suitable for vascular imaging. The contrast agent has good radiopacity, is inexpensive to produce, and is safe to handle. The material can be introduced into the vasculature of animal systems and provides good X-ray contrast. This provides a new method to image the fine vasculature of biological systems.

What is claimed is:

1. An injectable contrast agent comprising:
a nanoparticle, the nanoparticle comprising
a core comprising at least one of an abellaite (NaPb₂(CO₃)₂OH) core, a hydrocerussite (2PbCO₃—Pb(OH)₂) core, a lead (II) carbonate (PbCO₃) core, a lead (II) tungstate (PbWO₄) core, a bismuth oxide (Bi₂O₃) core, and combinations thereof; and
a capping agent comprising at least one of an oligomer and a polymer; and
a gel precursor solution.

2. The injectable contrast agent of claim 1, wherein the gel precursor solution is an alginate gel precursor solution.

3. The injectable contrast agent of claim 2, wherein the gel precursor solution comprises from about 0.01% (w/w) alginate to about 2% (w/w) alginate.

4. The injectable contrast agent of claim 1, wherein the capping agent is selected from the group consisting of tetraethylene glycol, triethethylene glycol, polyethylene-glycol, polyvinyl alcohol, polyacrylic acid, polyacrylamide, poly(sodium 4-styrenesulfonate), and combinations thereof.

5. The injectable contrast agent of claim 1, comprising from about 0.05 g/mL of the nanoparticle to about 1 g/mL of the nanoparticle.

6. The injectable contrast agent of claim 1, wherein the core diameter of the nanoparticle ranges from about 1 nm to about 500 nm.

7. The injectable contrast agent of claim 1, wherein the core of the nanoparticle is bismuth oxide (Bi₂O₃) core and the nanoparticle diameter ranges from about 1 nm to about 10 nm.

8. The injectable contrast agent of claim 1, wherein the gel precursor solution further comprises polyacrylic acid, polyvinyl alcohol, chitosan, and combinations thereof.

9. The injectable contrast agent of claim 1, wherein the gel precursor solution further comprises a chelating gelation control reagent selected from the group consisting of ethylenediaminetetraacetic acid, glucono-δ-lactone in combination with lead (II) carbonate, nitrilotriacetic acid, trans-1,2-diaminocylcohexanetetraacetic acid, diethlyenetriaminepentaacetic acid, bis(aminoethyl)glycolether-N,N,N′,N′-tetraacetic acid, calcium chloride, and combinations thereof.

10. A method for post mortem imaging vasculature of a subject, the method comprising:
introducing into the subject's vasculature an injectable contrast agent, the injectable contrast agent comprising
a nanoparticle, the nanoparticle comprising
a core comprising at least one of an abellaite (NaPb₂(CO₃)₂OH) core, a hydrocerussite (2PbCO₃—Pb(OH)₂) core, a lead (II) carbonate (PbCO₃) core, a lead (II) tungstate (PbWO₄) core, a bismuth oxide (Bi₂O₃) core, and combinations thereof; and
a capping agent comprising at least one of an oligomer and a polymer; and
a gel precursor solution, wherein the nanoparticle is dispersed in the gel precursor solution; and
imaging the subject using an X-ray technique.

11. The method of claim 10, wherein the gel precursor solution is an alginate gel precursor solution.

12. The method of claim 11, wherein the gel precursor solution comprises from about 0.01% (w/w) alginate to about 2% (w/w) alginate.

13. The method of claim 10, wherein the capping agent is selected from the group consisting of tetraethylene glycol, triethethylene glycol, polyethylene glycol, polyvinyl alcohol, polyacrylic acid, polyacrylamide, poly(sodium 4-styrenesulfonate), and combinations thereof.

14. The method of claim 10, wherein the injectable contrast agent comprises from about 0.05 g/mL of the nanoparticle to about 1 g/mL of the nanoparticle.

15. The method of claim 10, wherein the core diameter ranges from about 1 nm to about 500 nm.

16. The method of claim 10, wherein the core is the bismuth oxide (Bi₂O₃) core and ranges in diameter from about 1 nm to about 5 nm.

17. The method of claim 10, wherein the crosslinking ranges from about 10 seconds to about 48 hours at a temperature ranging from about 5° C. to about 40° C.

18. The method of claim 10, wherein the injectable contrast agent further comprises polyacrylic acid; polyvinyl alcohol, chitosan, and combinations thereof.

19. The method of claim 10, wherein the injectable contrast agent further comprises a chelating gelation control reagent selected from the group consisting of ethylenediaminetetraacetic acid, glucono-δ-lactone in combination with lead (II) carbonate, nitrilotriacetic acid, trans-1,2-diaminocylcohexanetetraacetic acid, diethlyenetriaminepentaacetic acid, and bis(aminoethyl)glycolether-N,N,N′,N′-tetraacetic acid, calcium chloride, and combinations thereof.

20. The method of claim 10, wherein the injectable contrast agent comprises a gelation time ranging from about 5 minutes to about 48 hours.
